SEQUENCE LISTING (33 sequences)

SEQ ID NO: 1 - protein, 122 aa, Homo sapiens [sequence omitted]

SEQ ID NO: 2 - protein, 107 aa, Homo sapiens
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 3 - protein, 107 aa, Homo sapiens
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIK

SEQ ID NO: 4 - protein, 451 aa, Homo sapiens
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 448 aa, Homo sapiens
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 214 aa, Homo sapiens
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, Homo sapiens
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1356 bp, Homo sapiens
  source 1..1356: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 9 - DNA, 1347 bp, Homo sapiens
  source 1..1347: mol_type = genomic DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 10 - DNA, 645 bp, Homo sapiens
  source 1..645: mol_type = genomic DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttactggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - DNA, 645 bp, Homo sapiens
  source 1..645: mol_type = genomic DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttagtggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 5 aa, Homo sapiens
RYYMY

SEQ ID NO: 13 - protein, 17 aa, Homo sapiens
EINPNNGGTKFNEKFKN

SEQ ID NO: 14 - protein, 13 aa, Homo sapiens
EDDYDGTPFAMDY

SEQ ID NO: 15 - protein, 11 aa, Homo sapiens
HASQNINVWLS

SEQ ID NO: 16 - protein, 7 aa, Homo sapiens
KASNLHT

SEQ ID NO: 17 - protein, 9 aa, Homo sapiens
QQGQTYPYT

SEQ ID NO: 18 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 19 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 20 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 21 - protein, 243 aa, Macaca fascicularis
MGSSLAFLLLSFHVCVLLLQLLMPHSAQFAVVGPPGPILAMVGEDADLPCHLFPTMSAETMELRWVSSNLRQVVNVYADGKEVEDRQSAAYRGRTSILRDGITAGKAALRIHNVTASDSGKYLCYFQDGDFYEKALVELKVAALGSDLHIDVKGYEDGGIHLECRSTGWYPQPQIRWSNDKGENIPAVEAPVFVDGVGLYAVAASVILRGSSGEGVSCTIRSSLLGLEKTTSISIAGHHHHHH

SEQ ID NO: 22 - protein, 243 aa, Macaca fascicularis
MGSSLAFLLLNFHVSFFLVQLLTPCSAQFSVLGPSGPILAMVGEDADLPCHLFPTMSAETMELRWVSSSLRQVVNVYADGKEVEDRQSAPYRGRTSILRDDIAAGKAALRIHNVTASDSGKYLCYFQDADFYEKALVELKVAALGSNLHVEVKGYEDGGIHLECRSTGWYPQPKIQWSNAKGQNIPAVEAPVVADGVGLYAVAASVIMRGGSGESVSCIIRNSVLGLEKTASISIADHHHHHH

SEQ ID NO: 23 - protein, 243 aa, Macaca fascicularis
MANFLAFLLLNFRVCLLLVQLLTPCSAQFAVLGPHGPILAMVGEDVDLPCHLFPTMSAETMELRWVSSSLRQVVNVYSDGKEVEDRQSAPYRGRTSILRDGITAGKAALRIHNVTASDSGKYLCYFQDGDFYEKALVELKVAALGSDLHIEVKGYEDGGIHLECRSTGWYPQPQIQWSNTKGQHIPAVKAPVVADGVGLYAVAASVIMRGSSGEGVSCIIRNSLLGLEKTASISITDHHHHHH

SEQ ID NO: 24 - protein, 122 aa, synthetic construct
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSS

SEQ ID NO: 25 - protein, 107 aa, synthetic construct
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIK

SEQ ID NO: 26 - protein, 5 aa, Mus musculus
RYYLY

SEQ ID NO: 27 - protein, 17 aa, Mus musculus
EINPNNGGTKFNEKFKS

SEQ ID NO: 28 - protein, 13 aa, Mus musculus
EDDYDGTPDAMDY

SEQ ID NO: 29 - protein, 11 aa, Mus musculus
HASQNINLWLS

SEQ ID NO: 30 - protein, 7 aa, Mus musculus
RASNLHT

SEQ ID NO: 31 - protein, 9 aa, Mus musculus
QQGHSYPYT

SEQ ID NO: 32 - protein, 452 aa, synthetic construct
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, synthetic construct
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0227558 A1
(43) Pub. Date: Jul. 20, 2023

(54) SELECTION OF RESPONDERS FOR ANTI-BTN3A TREATMENT

(71) Applicant: Imcheck Therapeutics SAS, Marseille (FR)

(72) Inventors: Emmanuel VALENTIN, Marseille Cedex 09 (FR); Paul FROHNA, Marseille Cedex 09 (FR); Aude DE GASSART, Marseille Cedex 09 (FR)

(21) Appl. No.: 18/183,588

(22) Filed: Mar. 14, 2023

Related U.S. Application Data
(63) Continuation-in-part of application No. PCT/EP2022/075608, filed on Sep. 15, 2022.

(30) Foreign Application Priority Data
Sep. 15, 2021 (EP) 21306272.2

(51) Int. Cl. C07K 16/28

(52) U.S. Cl. CPC C07K 16/2827; A61K 2039/505

(57) ABSTRACT

The present disclosure relates to the pharmaceutical field. More specifically, it relates to methods for treating a tumor in a human subject in need thereof, said method comprising administering a therapeutically efficient amount of an anti-BTN3A antibody which induces the activation of Vγ9Vδ2 T cells, in combination with a therapeutically efficient amount of an anti-PD1/PDL1 treatment, wherein said subject is having relapsed or refractory tumors to anti-PD1/PDL1 treatment.

The present disclosure relates to the pharmaceutical field. More specifically, it relates to methods of treating cancer disorders with activating anti-BTN3A antibodies.

### SEQUENCE LISTING

This document incorporates by reference an electronic sequence listing file, which was electronically submitted along with this document. The text file is named 2023-03-14_13500185PA_SeqList.xml, is 37960 bytes, and was created on Mar. 10, 2023.

### BACKGROUND

Peripheral blood is technically easy to obtain and process, can be repeatedly accessed over longer time periods and mostly provides a sufficient sample volume. For these reasons, peripheral blood is a highly-utilized material for immune population monitoring in tumor patients (Schnell et al, Biomedicines 2018, 6, 25). To the contrary, accessing and phenotyping the different immune cells that infiltrate the tumor tissues remains technically challenging (Maibach et al, 2020, Front Immunol 2020; 11:2105; Lara et al 2019, Nature, Scientific Reports 9:17589).

Considering the highly diverse, dynamic nature of the immune system, changes in phenotype, function and metabolism of peripheral immune cells are generally not considered representative of changes occurring within the tumors (Schnell et al, Biomedicines 2018, 6, 25; Maibach et al, 2020, Front Immunol 2020; 11:2105).

In addition, no clear correlation has been described between the frequency or absolute counts in the peripheral blood of different immune cells population and their respective infiltration in tumoral tissues. This is particularly true for innate immune cell population, such as NK cells or gamma delta T cells.

WO2020/025703 discloses anti-BTN3A antibodies which activate the cytolytic function, cytokine production and/or proliferation of Vγ9Vδ2 T cells (activating anti-BTN3A antibodies), and thereby may be used as immunotherapeutic agents to overcome the immunosuppressive mechanisms observed in cancer patients.

The inventors now provide evidence for a correlation between the absolute count of γδ T cells in the peripheral blood and activation of γδ T cells in tumoral tissues, thereby raising the possibility to select responders to treatment with activating anti-BTN3A antibodies, based on the absolute count of γδ T cells in the peripheral blood.

The inventors further provide, for the first time, evidence of clinical efficacy to a combination therapy with anti-BTN3A antibodies and anti-PD1 antibodies in patients having refractory or relapsed tumors, having received prior immunotherapies with anti-PD1 antibodies.

### SUMMARY

A first aspect of the disclosure relates to an isolated activating anti-BTN3A antibody which induces the activation of Vγ9Vδ2 T cells, for use in treating a tumor in a human subject in need thereof, wherein said subject has been selected for said treatment with said anti-BTN3A antibody by evaluating the blood baseline Vγ9Vδ2 T cell counts in said subject. In specific embodiments, said subject has been selected for said treatment when the baseline circulating Vγ9Vδ2 T cell counts is higher than 5000 cells/mL, higher than 10 000 cells/mL, or higher than 20 000 cells/mL.

Another aspect of the disclosure relates to an isolated activating anti-BTN3A antibody which induces the activation of Vγ9Vδ2 T cells, for use in treating a solid tumor in a human subject in need thereof, wherein said subject has been selected for said treatment with said anti-BTN3A antibody by evaluating the tumor baseline Vγ9+ T cell density in a solid tumor biopsy from said subject. In specific embodiments, said subject has been selected for said treatment when tumor baseline Vγ9+ T cell density is higher than 2, 3, 4, 5, 6, 7, 8, 9 or 10 cells/mm².

The disclosure also relates to a method for treating a tumor in a human subject in need thereof, which comprises administering a therapeutically efficient amount of an activating anti-BTN3A antibody, wherein said human subject has been selected for said anti-BTN3A activating antibody treatment, by evaluating the blood baseline Vγ9Vδ2 T cell counts in said subject.

The disclosure also relates to a method for treating a tumor in a human subject in need thereof, which comprises administering a therapeutically efficient amount of an activating anti-BTN3A antibody, wherein said human subject has been selected for said anti-BTN3A activating antibody treatment, by evaluating baseline Vγ9+ T cell density in a tumor biopsy of said subject.

Another aspect is directed to a method for selecting a subject eligible to an activating anti-BTN3A antibody treatment, said method comprising the step of evaluating the blood baseline Vγ9Vδ2 T cell counts in said subject.

Another aspect is directed to a method for selecting a subject eligible to an activating anti-BTN3A antibody treatment, said method comprising the step of evaluating the baseline Vγ9+ T cell density in a tumor biopsy of said subject.

The disclosure further relates to a method for treating a tumor in a human subject in need thereof, which comprises administering a therapeutically efficient amount of an activating anti-BTN3A which induces the activation of Vγ9Vδ2 T cells, in combination with a therapeutically efficient amount of an anti-PD1/PDL1 treatment, wherein said subject is having relapsed or refractory tumors after prior anti-PD1/PDL1 treatment.

Further disclosed herein is a method for treating a tumor in a human subject in need thereof, which comprises administering a therapeutically efficient amount of an activating anti-BTN3A which induces the activation of Vγ9Vδ2 T cells, wherein said tumor is a solid tumor selected from the group consisting of bladder cancer, melanoma, non small cell lung cancer, and head and neck squamous cell carcinoma.

In preferred embodiments, said activating BTN3A antibody is mAb1 as described herein or a functional variant thereof having the 6 CDRs of mAb1.

### BRIEF DESCRIPTION OF THE FIGURES

FIG. 1A-C: In EVICTION, immune activation and IFNg production are related to both ICT01 dose and baseline circulating Vγ9V62 T cell absolute counts. Baseline absolute number (per milliliter of blood) of Vγ9Vδ2 T cells and activation markers (CD69 and PD-L1, D0+24h) in blood of patients assessed by flow cytometry. ICT01 Cmax assayed by an ultra-sensitive PK assay (Chimera Biotec, Germany). Circulating cytokine levels assessed by commercial kits (MesoScale Discovery). IFNγ Fold Change defined as the ratio between IFNγ AUC (using D0, D+30 min, D0+4h and D+24h timepoints) and IFNγ baseline AUC. Spearman correlation. Panel A: % of CD69 positive NK cells, panel B: % of PD-L1 positive granulocyte, panel C: IFNγ fold change.

FIG. 2A-E: In EVICTION, increase in tumoral immune infiltration and activation post ICT01 is related to baseline γ9δ2 T cell count. Panel A: Baseline absolute number (per milliliter of blood) of Vγ9Vδ2 T cells in blood of patients before ICT01 administration. Panel B: Log 10FoldChange (post-treatment vs pre-treatment biopsies) of tumoral cell densities (cell/mm2) of the different phenotypes assessed by multiplex quantitative IHC (digital pathology, Veracyte, France) in all patients from group A and C with available good quality biopsy pairs. Panel C to E: Log 10FoldChange (post-treatment vs pre-treatment biopsies) of tumoral cell densities (cell/mm2) of the different phenotypes assessed by multiplex quantitative IHC (digital pathology, Veracyte, France). The patients population is divided according to baseline absolute number of Vγ9Vδ2 T cells, 20000 cell/mL cut-off.

FIG. 3: Vγ9TCR+ Cells at the tumor are related to circulating Vγ9V52 T cell counts. Baseline tumoral cell density of Vγ9TCR+ assessed by digital pathology, quantitative IHC (cell/mm2). Baseline absolute number (per milliliter of blood) of Vγ9Vδ2 T cells in blood of patients before ICT01 administration. Spearman correlation.

FIG. 4A-B: ICT01 increases PD-1 expression in EVICTION patients. ICT01-induced activation of Vγ9Vδ2 T cells increases surface expression of PD-1 (Flow cytometry on frozen biopsies, mean per dose cohort) in cancer patients treated in Group A (FIG. 4A) and Group B (FIG. 4B) of EVICTION. Two-way ANOVA and Holm-Šidák's multiple comparisons test

FIG. 5A-D: ICT01/Pembrolizumab treatment induces activation and migration of multiple immune cell populations. Baseline absolute number (per milliliter of blood, % of baseline) and activation markers (CD69 and PD-L1, % of positivity) in blood of patients assessed by flow cytometry at different time points. Panel A: Vγ9Vδ2 T cells, panel B: NK cells, panel C: CD8 T cells, and panel D: granulocytes.

FIG. 6A: Cell Densities (number of cell per mm²) of CD3+ T cells and CD3+Ki67+ T cells were evaluated in pre-treatment and post-treatment tumor biopsies using digital pathology and % of change of Ki67+ cells among CD3+ cells were computed. Patients were classified according to the composite Cmax X baseline circulating Vγ9Vδ2 T cells count.

FIG. 6 B: Cell Densities (number of cell per mm²) of CD3+CD8+ T cells and CD3+CD8+Ki67+ T cells were evaluated in pre-treatment and post-treatment tumor biopsies using digital pathology and % of change of Ki67+ cells among CD3+CD8+ cells were computed. Patients were classified according to the composite Cmax X baseline circulating Vγ9Vδ2 T cells count.

FIG. 6 C: Cell Densities (number of cell per mm²) of CD3+CD8+ T cells, CD3+CD8+PD-1+, CD3+CD8+LAG3+, CD3+CD8+ TIM3+ T cells were evaluated in pre-treatment and post-treatment tumor biopsies using digital pathology and % of change of triple negative (PD-1 negative, LAGS negative and TIM3 negative) CD3+CD8+ T cells among CD3+CD8+ cells were computed. Patients were classified according to the composite Cmax X baseline circulating Vγ9Vδ2 T cells count.

FIG. 7A: % of PD-L1 positive tumor cells was evaluated in pre-treatment and post-treatment tumor biopsies by a trained pathologist and Log 2 Fold Change were computed. Patients were classified according to the composite ICT01 Cmax X baseline circulating Vγ9Vδ2 T cells count.

FIG. 7 B: % of PD-L1 positive immune cells was evaluated in pre-treatment and post-treatment tumor biopsies by a trained pathologist and Log 2 Fold Change were computed.

Patients were classified according to the composite ICT01 Cmax X baseline circulating Vγ9Vδ2 T cells count.

### DETAILED DESCRIPTION

### Definitions

In order that the present disclosure may be more readily understood, certain terms are first defined. Additional definitions are set forth throughout the detailed description.

As used herein, the term “BTN3A” has its general meaning in the art. In specific embodiments, it refers to human BTN3A polypeptides including either BTN3A1 of SEQ ID NO:18, BTN3A2 of SEQ ID NO:19 or BTN3A3 of SEQ ID NO:20.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, Sequences of Proteins of Immunological Interest, DIANE Publishing, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, an anti-BTN3A antibody is an antibody that binds specifically to BTN3A polypeptide.

As used herein, the term “binds specifically to” refers to the ability of an antibody to detectably bind an epitope presented on an antigen, such as a BTN3A polypeptide. In some embodiments, it is intended to refer to an antibody that binds to human BTN3A as expressed on peripheral blood marrow cells (PBMCs), preferably with an EC50 below 50 μg/ml and more preferably below 10 μg/ml as measured in a binding assay by flow cytometry as described in the Examples below. In other embodiments, it is intended to refer to an antibody that binds to an antigen (e.g. BTN3A polypeptide) with a K_{D} of 100 nM or less, 10 nM or less, 1 nM or less, 100 pM or less, or 10 pM or less, as measured by multicycle kinetics analysis as described in the Examples.

An “isolated antibody”, as used herein, refers to an antibody that is substantially free of other antibodies having different antigenic specificities (e.g., an isolated antibody that specifically binds to BTN3A is substantially free of antibodies that specifically bind to other antigens than BTN3A). An isolated antibody that specifically binds to BTN3A may, however, have cross-reactivity to other antigens, such as related BTN3A molecules from other species. Moreover, an isolated antibody may be substantially free of other cellular material and/or chemicals.

The term “monoclonal antibody” or “monoclonal antibody composition” as used herein refers to a preparation of antibody molecules of single molecular composition. A monoclonal antibody composition displays a single binding specificity and affinity for a particular epitope.

The phrases “an antibody recognizing an antigen” and “an antibody having specificity for an antigen” are used interchangeably herein with the term “an antibody which binds specifically to an antigen”.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction.

The term “K_{D}”, as used herein, is intended to refer to the dissociation constant, which is obtained from the ratio of Kd to Ka (i.e. Kd/Ka) and is expressed as a molar concentration (M). K_{D} values for antibodies can be determined using methods well established in the art. A method for determining the K_{D} of an antibody is by using surface plasmon resonance, or using a biosensor system such as a Biacore® system.

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN3A polypeptide). The term “affinity”, as used herein, means the strength of the binding of an antibody to an epitope.

As used herein, the term “activating antibody” refers to an antibody able to directly or indirectly induce immune functions of effector cells. In particular, as used herein, an activating anti-BTN3A antibody has at least the capacity to induce the activation of γδ T cells, typically Vγ9Vδ2 T cells, in co-culture with BTN3 expressing cells, with an EC50 below 5 μg/ml, preferably of 1 μg/ml or below, as measured in a degranulation assay as described in the Examples below.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. In preferred embodiments, the subject is a human subject.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, or the reduction of the size of the tumor.

As used herein, “blood Vγ9Vδ2 T cell counts” refers to the absolute number of Vγ9Vδ2 T cells circulating in a certain volume of a blood sample of a subject, as determined by standard Flow Cytometry methods associated with calibration beads, for example as described in the Examples.

As used herein, “tumor Vγ9+ T cell density” refers to the number of cells per mm² in a certain volume of a tumor biopsy of a subject, as determined by immunohistochemistry with an antibody that specifically binds to Vγ9TCR, such as the monoclonal antibody 7B6, for example as described in the Examples.

As used herein, “combination therapy”, “co-administration”, “combined administration” or “concomitant administration” refers to a combined administration of at least two therapeutic agents, where a first agent, typically an activating anti-BTN3A antibody (e.g. mAb1) is administered at the same time or separately within time intervals, with a second agent, for example an anti-PD-1 antibody (e.g pembrolizumab), in the same subject in need thereof, where these time intervals allow that the combined partners show a cooperative or synergistic effect for treating a disorder, e.g. a cancer. It is not intended to imply that the therapeutic agents must be administered at the same time and/or formulated for delivery together although these methods of delivery are within the scope described herein. The activating anti-BTN3A antibody can be administered concurrently with or prior to, or subsequent to one or more other additional therapies or therapeutic agents. The terms are also meant to encompass treatment regimens in which the agents are not necessarily administered by the same route of administration.

### The Subject in Need of an Activating Anti-BTN3A Antibody Treatment

Activating anti-BTN3A antibodies can activate the cytolytic function, cytokine production and/or proliferation of Vγ9Vδ2 T cells, and thereby may be used to overcome the immunosuppressive mechanisms observed in cancer patients (WO2020/025703).

As used herein, the term “cancer” refers to hyperproliferative and neoplastic disease states with cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The cancers may typically be classified in solid tumor cancers (non-hematologic malignancies) and hematologic malignancies.

Examples of hematological malignancies include, but are not limited to, B-cell lymphoid neoplasm, T-cell lymphoid neoplasm, non-Hodgkin lymphoma (NHL), B-NHL such as Diffule large B cell lymphoma (DLBLC), T-NHL, chronic lymphocytic leukemia (CLL), small lymphocytic lymphoma (SLL), mantle cell lymphoma (MCL), NK-cell lymphoid neoplasm and myeloid cell lineage neoplasm including acute myeloid leukemia.

Examples of non-hematological cancers include, but are not limited to, colorectal cancer, breast cancer, lung cancer (e.g. NSCLC), brain cancer, prostate cancer, head and neck cancer (e.g. HNSCC), pancreatic cancer, bladder cancer, colorectal cancer, bone cancer, cervical cancer, ovarian cancer, liver cancer, oral cancer, esophageal cancer, thyroid cancer, kidney cancer, stomach cancer, testicular cancer, urothelial cancer and skin cancer (e.g. melanoma).

In specific embodiment, said subject in need of an anti-BTN3A activating antibody treatment is having relapsed/refractory solid tumors. In more specific embodiment, said subject is having advanced stage, relapsed/refractory solid tumors.

In more specific embodiments, said subject is having relapsed/refractory solid tumors after a treatment with anti-PD1 or anti-PDL1 agent, such as an anti-PD1 or anti-PDL1 antibody, typically, ipilimumab, nivolumab or pembrolizumab. In more specific embodiments, said subject, having relapsed/refractory solid tumors after a treatment with anti-PD1 or anti-PDL1 agent, is also selected among subjects having bladder cancer, melanoma, non small cell lung cancer, and/or head and neck squamous cell carcinoma.

In other specific embodiments which may be combined with the previous embodiments, said subject in need of an anti-BTN3A activating antibody treatment is suffering from ovarian cancer.

### Activating Anti-BTN3A Antibody Treatment

As used herein, an “activating anti-BTN3A antibody treatment” relates to any therapeutic treatment comprising, administering, as the active principle, a therapeutically efficient amount of an activating anti-BTN3A antibody, optionally in combination with other therapeutic compounds.

Preferred examples of such activating anti-BTN3A antibody treatments are described in WO2012/80351 and WO2020/025703.

In specific embodiments, the activating anti-BTN3A antibody may be administered as the sole active ingredient or in conjunction with, other drugs e.g. for the treatment or prevention of diseases mentioned above.

In specific embodiments, the activating anti-BTN3A antibody may be administered in combination with anti-neoplastic agents.

In other specific embodiments, the activating anti-BTN3A antibody may be administered in combination with cell therapy (in particular γδ T cell therapy).

In other specific embodiments, the activating anti-BTN3A antibody may be administered in combination with immuno-cytokines (in particular, IL-2, IL-15, IL-21, IL-12, GM-CSF).

In other specific embodiments, the activating anti-BTN3A antibody may be administered with immunotherapeutic drugs, such as immune checkpoint inhibitors (in particular, anti-PD-1, anti-PD-L1, anti-CTLA-4, anti-TIGIT, anti-LAG-3, anti-TIL-3 antibody or other anti-PD1 or anti-PD-L1 agent).

As used herein, the term “cell therapy” refers to a therapy comprising the in vivo administration of at least a therapeutically efficient amount of a cell composition to a subject in need thereof. The cells administered to the patient may be allogenic or autologous. The term “γδ T cell therapy” refers to a cell therapy wherein the cell composition includes, as the active principle, γδ T cells, in particular Vγ9/Vδ2 T cells. In specific embodiments, said Vγ9/Vδ2 T cells have been expanded and/or activated ex vivo with a γδ T cell agonist.

A cell therapy product refers to the cell composition which is administered to said patient for therapeutic purposes. Said cell therapy product include a therapeutically efficient dose of cells and optionally, additional excipients, adjuvants or other pharmaceutically acceptable carriers.

Examples of antineoplastic agents which may be administered in combination with an activating anti-BTN3A antibody (e.g. mAb1 as described below) may include without limitation, alkylating agents (such as cyclophosphamide, mechloretamine, chlorambucil, melphalan, nitrosureas, temozolomide), anthracyclines (such as daunorubicin, doxorubicin, epirubicin, idarubicin, mitoxantrone, valrubicin), taxanes (such as Paclitaxel, Docetaxel), epothilones, inhibitors of Topoisomerase I (such as Irinotecan or Topotecan), inhibitors of Topoisomerase II (such as Etoposide, teniposide, or Tafluposide), nucleotide analogs and precursor analogs (such as azacitidine, azathioprine, capecitabine, cytarabine, flurouracil, gemcitabine, hydroxyurea, mercaptopurine, methotrexate, or Tioguanine), peptide antibiotics (such as carboplatin, cisplatin and oxaliplatin), retinoids (such as tretinoin, alitretinoin, bexarotene), vinca alkaloids and derivatives (such as vinblastine, vincristine, vindesine, vinorelbine), targeted therapies such as kinase inhibitors (such as Ibrutinib, Idelalisib, Erlotinib, Gefitinib, Imatinib, Vemurafenib, Vismodegib), proteasome inhibitors (such as bortezomib, carfilzomib), histone deacetylase inhibitors (such as Vorinostat or Romidepsin).

Examples of immunotherapeutic agents which may be administered in combination with an activating anti-BTN3A antibody (e.g. mAb1 as described below) include, without limitation, phosphoantigens (e.g. zoledronic acid or other bisphosphonates), anti-PD-1 antibodies, anti-PD-L1 antibodies, anti-BTLA antibodies, anti-CTLA-4 antibodies and cytokines (such as interleukin 2 (IL-2) (Choudhry H et al, 2018, Biomed Res Int. 2018 May 6), interleukin 15 (IL-15) (Patidar M et al., Cytokine Growth Factor Rev. 2016 Oct.; 31:49-59), interleukin 21 (IL-21) (Caccamo N. et al., PLoS One. 2012; 7(7):e41940), or interleukin 33 (IL-33) (Duault C et al., J Immunol. 2016 Jan. 1; 196(1):493-502)), or their recombinant forms and their derivatives, or any cytokines capable of inducing lymphocyte activity (e.g. proliferation or cytokines production or metabolic changes). The term derivative is used for any cytokine modifications that can rely on PEGylation (e.g. conjugation to polyethylene glycol (PEG) chains), mutation such as amino acid deletion, substitution or insertion, or association with potentiating agents (for example ID 5/IL15Ra complexes fused to an IgG1 Fc, in which IL-15 is additionally mutated (asn72asp) that further increase biological activity making this complex an IL-2 and IL-15Rβγ superagonist (Rhode P R et al, Cancer Immunol Res. 2016; 4(1):49-60)) (Barroso-Sousa Ret al, Curr Oncol Rep. 2018 Nov. 15; 21(1):1).

The term “IL-2” has its general meaning and refers to the human interleukin-2. IL-2 is part of the body's natural immune response. IL-2 mainly regulates lymphocyte activity by binding to IL-2 receptors.

The term “IL-15” has its general meaning and refers to the human interleukin-15. Like IL-2, IL-15 binds to and signals through a complex composed of IL-2/IL-15 receptor beta chain (CD122) and the common gamma chain (gamma-C, CD132). IL-15 regulates the activation and proliferation of T and natural killer (NK) cells.

The term “IL-21” has its general meaning and refers to the human interleukin-21. IL-21 has been ascribed to pleiotropic properties, including, but not limited to, enhancing NK cell and CD8+ T cell cytotoxicity, modulating plasma cell differentiation and inhibiting Treg cells.

The term “IL-33” has its general meaning and refers to the human interleukin-33. IL-33, considered as an alarmin released upon tissue stress or damage, is a member of the IL-1 family and binds the ST2 receptor. IL-33 is known as an effective stimulator of T_{H}1 immune cells, natural killer (NK) cells, iNKT cells, and CD8 T lymphocytes.

The term “PD-1” has its general meaning in the art and refers to the programmed death-1 receptor. The term “PD-1” also refers to a type I transmembrane protein, belonging to the CD28-B7 signalling family of receptors that includes CD28, cytotoxic T-lymphocyte-associated antigen 4 (CTLA-4), inducible costimulator (ICOS), and B- and T-lymphocyte attenuator (BTLA) (Greenwald R J et al., 2005, Annual Review of Immunology Vol 23 pp 515-548).

The term “anti-PD-1 antibody” or “anti-PD-1 agent” or “anti-PD-L1” has its general meaning in the art and refers to an antibody or other binding compound with binding affinity to PD-1 or PD-L1 respectively, and antagonist activity to PD-1, i.e., it inhibits the signal transduction cascade related to the PD-1 and inhibits PD-1 ligand binding (PD-L1; PD-L2). Such anti-PD-1 antibody/agent or anti-PD-L1 antibody/agent preferentially inactivates PD-1 with a greater affinity and potency, respectively, than its interaction with the other sub-types or isoforms of the CD28-B7 signalling family of receptors (CD28; CTLA-4; ICOS; BTLA). Tests and assays for determining whether a compound is a PD-1 antagonist are well known by the skilled person in the art such as described in Shaabani S, et al (2015-2018). Expert Opin Ther Pat. 2018 Sep.; 28(9):665-678; Seliger, B. J. Clin. Med. 2019, 8, 2168.

Examples of such anti-PD1 or anti-PDL1 antibody includes without limitation, nivolumab, pembrolizumab, avelumab, durvalumab, cemiplimab, or atezolizumab.

In specific embodiments, an “anti-PD1/PD-L1 treatment” includes administering a therapeutically effective amount of an anti-PD-1 or anti-PD-L1 agent, in particular an anti-PD-1 or anti-PD-L1 antibody in a subject in need thereof.

Examples of such anti-CTLA4 antibody includes without limitation, ipilimumab.

In specific embodiments, an “activating anti-BTN3A antibody treatment” includes a method as defined above comprising co-administration, e.g. concomitantly or in sequence, of a therapeutically effective amount of an activating anti-BTN3A antibody as defined herein, and at least one second drug substance, said second drug substance being an immunotherapeutic agent (such as anti-PD-1, anti-PD-L1 antibodies or other binding compounds), and/or a cytokine, e.g. IL-2 or IL-15, or their derivatives, e.g. as indicated above. In specific embodiments, an activating anti-BTN3A antibody treatment comprises co-administration of a therapeutically effective amount of an anti-BTN3A activating antibody as defined herein, and a therapeutically effective amount of an anti-PD-1 or anti-PD-L1 agent, such as anti-PD-1 or anti-PD-L1 antibody. In other specific embodiments, an activating anti-BTN3A antibody treatment comprises co-administration, e.g. concomitantly or in sequence, of a therapeutically effective amount of an anti-BTN3A activating antibody as defined herein, and a therapeutically effective amount of a IL-2 or IL-15 or their derivatives, pegylated variants and superagonist variants thereof, e.g. as indicated above, optionally in combination with an anti-PD1 antibody, such as pembrolizumab.

In specific embodiments, the activating anti-BTN3A antibodies are formulated in a pharmaceutical composition. For example, the pharmaceutical composition may include one or more additional excipients, including buffers, stabilizing agents, antioxydants, surfactants, or salts.

According to an embodiment, the pharmaceutical composition comprising the activating anti-BTN3A antibody is an aqueous solution, for example an injectable formulation.

According to a particular embodiment, the pharmaceutical composition comprising the activating anti-BTN3A antibody is a solution for infusion. Antibody formulations for intravenous or subcutaneous administration are well-known in the art and for example described in Razinkov et al. J Biomol Screen. 2015 April; 20(4):468-83.

The pharmaceutical composition comprising the activating anti-BTN3A antibody can be formulated at various concentrations. For example, the formulation may comprise the activating anti-BTN3A antibody at a concentration of between 0.1 μM and 1 mM, more preferably between 1 μM and 500 μM, between 500 μM and 1 mM, between 300 μM and 700 μM, between 1 μM and 200 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the activating anti-BTN3A antibody at a concentration of between 300 μM and 700 μM.

Typically, the therapeutic dose of the activating anti-BTN3A antibody in a human patient will be in the range of 100 μg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly, every three weeks or monthly.

Typically, in specific embodiments, said activating anti-BTN3A antibody is administered intravenously at least twice at a dose comprised between 7 and 200 mg each dose, typically every 21 days.

For example, suitable dose for intravenous administration of an activating anti-BTN3A antibody (preferably mAb1 as described hereafter) as a monotherapy are selected from 7, 10, 20, 50, 75, 100, 125, 150, 175 and 200 mg. In other specific embodiments, suitable dose for intravenous administration of an activating anti-BTN3A antibody (preferably mAb1) as a monotherapy are selected from 7, 10, 20, 50, 75, 100, 125, 150, 175 and 200 mg. In more specific embodiments, a suitable dose for intravenous administration of an activating anti-BTN3A antibody (preferably mAb1) as a monotherapy is selected from 20 and 75 mg per administration.

In specific embodiments where said activating anti-BTN3A antibody is administered in combination with an anti-PD1 or anti-PD-L1 antibody, suitable dose for intravenous administration of an activating anti-BTN3A antibody is selected from 7, 10, 20, 50, 75, 100, 125, 150, 175 and 200 mg. Anti-PD1 or anti-PD-L1 antibody may be administered according to the approved dose of the manufacturer. For example, in specific embodiments of the methods of the disclosure, Pembrolizumab (KEYTRUDA) may be administered in combination with said activating anti-BTN3A antibody (preferably mAb1), typically Pembrolizumab is administered by intravenous infusion at a dose of 200 mg and mAb1 is administered at a dose between 20 and 70 mg.

In specific embodiments, the activating anti-BTN3A antibody for use according to the methods of the disclosure (preferably mAb1 as described hereafter) is administered intravenously at least twice at a dose comprised between 7 and 200 mg each dose, preferably the second dose being administered at least 15 days after the first dose, typically after about 21 days.

### Preferred Activating Anti-BTN3A Antibodies

In specific embodiments, said activating anti-BTN3A antibody for use according to the present methods of the disclosure have one or more of the following properties:
(i) it binds to BTN3A with a K_{D} of 10 nM or less, preferably with a K_{D} of 1 nM or less, as measured by SPR, for example as described in the Examples below;
(ii) it cross-reacts to cynomolgus BTN3A1 of SEQ ID NO:21, BTN3A2 of SEQ ID NO:22 and/or BTN3A3 of SEQ ID NO:23 with a K_{D} of 100 nM or less, preferably with a K_{D} of 10 nM or less, as measured by SPR, for example as described in the Examples below;
(iii) it binds to human PBMCs with an EC₅₀ of 50 μg/ml or below, preferably of 10 μg/ml or below, as measured in a flow cytometry assay as described in the Examples below;
(iv) it induces the activation of γδ-T cells, typically Vγ9Vδ2 T cells, in co-culture with BTN3 expressing cells, with an EC₅₀ below 5 μg/ml, preferably of 1 μg/ml or below, as measured in a degranulation assay as described in the Examples below.

In specific embodiments, the activating anti-BTN3A antibody for use in the methods of the present disclosure is a humanized antibody. Typically, a non-human antibody is humanized to reduce immunogenicity to humans, while having at least the same affinity (or superior affinity) of the parental non-human antibody. In specific embodiments, the activating anti-BTN3A antibody is a humanized antibody of the parent antibody mAb 7.2 as disclosed in WO2012/080351. Other examples include humanized antibodies of the parent antibody mAb 20.1 as disclosed in WO2012/080351.

Generally, a humanized antibody comprises one or more variable domains in which, CDRs, (or portions thereof) are derived from a non-human antibody, e.g. the murine mAb 7.2 or mAb 20.1, and framework regions (FRs) (or portions thereof) are derived from the murine antibody sequences with mutations to reduce immunogenicity.

A humanized antibody optionally will also comprise at least a portion of a human constant region.

In specific embodiments, the activating anti-BTN3A antibody according to the disclosure is a humanized silent antibody, typically a humanized silent IgG1 or IgG4 antibody.

As used herein, the term “silent” antibody refers to an antibody that exhibits no or low FcγR binding and/or C1q binding as measured in binding assays such as those described in the Examples.

In one embodiment, the term “no or low FcγR and/or C1q binding” means that the silent antibody exhibits an FcγR and/or C1q binding that is at least below 50%, for example below 80% of the FcγR and/or C1q binding that is observed with the corresponding antibody with wild type human IgG1 or IgG4 isotype.

Preferred antibodies for use in the methods of the present disclosure comprises the use of antibodies with heavy and light chains including the following 6 CDRs of murine mAbs 7.2 antibody (also disclosed in WO2012/80769).

The amino acid sequences of the VH CDR1s (also called HCDR1), VH CDR2s (also called HCDR2), VH CDR3s (also called HCDR1), VL CDR1s (also called LCDR1), VL CDR2s (also called LCDR2), VL CDR3s (also called HCDR3) of mAb7.2 and of mAb20.1 are shown in Table 1, the CDR regions bing delineated using the Kabat numbering (Kabat et al., 1992, hereafter “Kabat et al.”).

For the ease of reading, the CDR regions are called hereafter HCDR1, HCDR2, HCDR3, LCDR1, LCDR2, LCDR3 respectively.

TABLE 1
CDR regions murine mAb 7.2 and mAb 20.1
antibody according to Kabat numbering

| Original | | | | | | |
|---|---|---|---|---|---|---|
| antibody | HCDR1 | HCDR2 | HCDR3 | LCDR1 | LCDR2 | LCDR3 |
| mAb 7.2 | SEQ ID | SEQ ID | SEQ ID | SEQ ID | SEQ ID | SEQ ID |
| | NO: 12 | NO: 13 | NO: 14 | NO: 15 | NO: 16 | NO: 17 |
| mAb 20.1 | SEQ ID | SEQ ID | SEQ ID | SEQ ID | SEQ ID | SEQ ID |
| | NO: 26 | NO: 27 | NO: 28 | NO: 29 | NO: 30 | NO: 31 |

More preferred antibodies include humanized antibodies having the 6 CDRs of mAb 7.2 or mAb 20.1 as disclosed in Table 1.

In other specific embodiments, the methods of the present disclosure comprise the use of antibodies with heavy and light chains including the CDRs of mAb 20.1 antibody as disclosed in WO2012/80769. In particular, in more specific embodiments, the methods of the present disclosure comprise the use of humanized antibodies of parent murine mAb 20.1 and including the CDRs of mAb 20.1 antibody.

Humanized antibodies as used in the methods of the disclosure may include modifications made to framework residues within VH and VL, to decrease the immunogenicity of the antibody as compared to the corresponding murine antibodies, typically as compared to corresponding framework regions of mAb 7.2 or mAb 20.1.

In one specific embodiment, the antibody of the disclosure is a humanized monoclonal antibody of the parent murine antibody mAb 7.2, having the 6 CDRs of mAb 7.2 and further including at least the following amino acid mutations in the VH framework regions: V5Q; V11L; K12V; R66K; S74F; 175S; E81Q; S82AR; R82BS; R83T; D85E; T87S; L108S; and at least the following amino acid mutations in the Vκ framework regions: T5N; V15L; R18T; V19I; K42N; A43I; D70G; F73L; Q100G; when compared to the original murine framework regions of parental murine mAb 7.2.

In another specific embodiment, the antibody of the disclosure is a humanized monoclonal antibody of the parent murine antibody mAb 7.2, including at least the following amino acid mutations in the VH framework regions as compared to mAb 7.2: V5Q; V11 L; K12V; R66K; S74F; 175S; E81Q; S82AR; R82BS; R83T; D85E; T87S; L108S; and at least the following amino acid mutations in the Vκ framework regions: T5N; V15L; R18T; V19I; K42N; A43I; S63T; D70G; F73L; Q100G when compared to the original murine framework regions of parental murine mAb 7.2.

In more preferred embodiment, the methods of the present disclosure comprise the use of one of the following humanized recombinant antibodies selected from the group consisting of mAb1, mAb2, mAb3, mAb4, and mAb5 and comprising the variable heavy and light chain amino acid sequences and, optionally the human constant regions (isotypes), as described in the Table 2 below:

TABLE 2
Variable heavy and light chain amino
acid sequences of mAb1-mAb4

| | VH | VL | |
|---|---|---|---|
| | Amino acid | Amino acid | Isotype constant |
| Antibody | sequence | sequence | region |
| mAb1 | SEQ ID NO: 1 | SEQ ID NO: 2 | Silent IgG1 |
| | (VH2 7.2) | (Vk1 7.2) | L247F/L248E/P350S |
| mAb2 | SEQ ID NO: 1 | SEQ ID NO: 3 | Silent IgG1 |
| | (VH2 7.2) | (Vk2 7.2) | L247F/L248E/P350S |
| mAb3 | SEQ ID NO: 1 | SEQ ID NO: 2 | IgG4 S241P/L248E |
| | (VH2 7.2) | (Vk1 7.2) | |
| mAb4 | SEQ ID NO: 1 | SEQ ID NO: 3 | IgG4 S241P/L248E |
| | (VH2 7.2) | (Vk2 7.2) | |
| mAb5 | SEQ ID NO: 24 | SEQ ID NO: 25 | Silent IgG1 |
| | (humanized VH | (humanized VL | L247F/L248E/P350S |
| | mAb20.1) | mAb20.1) | |

The corresponding amino acid and nucleotide coding sequence of the constant isotype regions of IgG1, IgG4 and their mutant versions IgG1 L247F/L248E/P350S and IgG4 S241P/L248E used for generating mAb1 to mAb4 and mAb5 are well-known in the art (Oganesyan et al., 2008; Acta Crystallogr. D Biol. Crystallogr. 64, 700-704; Reddy et al., 2000; J. Immunol. 164, 1925-1933). The C-terminal lysine found in IgG may be naturally cleaved off and this modification does not affect the properties of the antibody; so, this residue may additionally be deleted in the constructs of mAb1 to mAb4 and mAb5.

Full length light and heavy chains and corresponding coding sequences of mAb1, mAb2, mAb3, mAb4 and mAb5 are shown in the Table 3 below.

TABLE 3
Full length heavy and light chain DNA coding sequences

| Antibody | Amino acid sequence | DNA coding sequence |
|---|---|---|
| mAb1 | Heavy Chain: SEQ ID NO: 4 | Heavy Chain: SEQ ID NO: 8 |
| | Light Chain: SEQ ID NO: 6 | Light Chain: SEQ ID NO: 10 |
| mAb2 | Heavy Chain: SEQ ID NO: 4 | Heavy Chain: SEQ ID NO: 8 |
| | Light Chain: SEQ ID NO: 7 | Light Chain: SEQ ID NO: 11 |
| mAb3 | Heavy Chain: SEQ ID NO: 5 | Heavy Chain: SEQ ID NO: 9 |
| | Light Chain: SEQ ID NO: 6 | Light Chain: SEQ ID NO: 10 |
| mAb4 | Heavy Chain: SEQ ID NO: 5 | Heavy Chain: SEQ ID NO: 9 |
| | Light Chain: SEQ ID NO: 7 | Light Chain: SEQ ID NO: 11 |
| mAb5 | Heavy Chain: SEQ ID NO: 32 | — |
| | Light Chain: SEQ ID NO: 33 | |

In certain embodiments that may be combined with the previous embodiments, an antibody provided herein is an antibody fragment of the above-defined antibodies.

As used herein, the term antibody fragments include any fragment of an antibody maintaining the antigen-binding region with substantially the same functional properties with respect to BTN3A target as the original antibody.

Antibody fragments include, but are not limited to, Fab, Fab′, Fab′-SH, F(ab′)2, Fv, Unibody, and scFv fragments, diabodies, single domain or nanobodies and other fragments.

In specific embodiments, said antibody fragment is a monovalent antibody, such as a Fab of scFv fragments.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Single-domain antibodies are antibody fragments comprising all or a portion of the heavy chain variable domain or all or a portion of the light chain variable domain of an antibody.

In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells as described herein.

### Methods for Producing the Activating Anti-BTN3A Antibodies

Activating anti-BTN3A antibodies for use in the methods of the present disclosure can be produced in a host cell transfectoma using, for example, a combination of recombinant DNA techniques and gene transfection methods as is well known in the art (Morrison, 1985 Science 229, 1202-1207).

In one specific embodiment, a cloning or expression vector according to the disclosure comprises one of the coding sequences of the heavy and light chains of any one of mAb1, mAb2, mAb3, mAb4 or mAb5, operatively linked to suitable promoter sequences.

Mammalian host cells for expressing the recombinant antibodies of the disclosure include Chinese Hamster Ovary (CHO cells) including dhfr-CHO cells (described in Urlaub and Chasin, 1980, Proc. Natl. Acad. Sci. U.S.A 77, 4216-4220) used with a DHFR selectable marker (as described in Kaufman and Sharp, 1982 Mol. Cell. Biol. 2, 1304-1319), CHOK1 dhfr+ cell lines, NSO myeloma cells, COS cells and SP2 cells, for example GS CHO cell lines together with GS Xceed™ gene expression system (Lonza). When recombinant expression vectors encoding antibody genes are introduced into mammalian host cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007, J. Chromatogr. B 848, 28-39).

In one specific embodiment, the host cell of the disclosure is a host cell transfected with an expression vector having the coding sequences suitable for the expression of mAb1, mAb2, mAb3, mAb4 and mAb5 respectively, operatively linked to suitable promoter sequences.

For example, a host cell comprising at least the nucleic acids of SEQ ID NO:8 and 10, encoding respectively the heavy and light chains of mAb1, is used for producing an antibody for use according to the present methods of the disclosure.

The latter host cells may then be further cultured under suitable conditions for the expression and production of an activating anti-BTN3A antibody, typically selected from the group consisting of mAb1, mAb2, mAb3, mAb4 and mAb5 respectively.

Alternatively, cell free expression systems may be used for the production of any activating anti-BTN3A antibody, for example mAb1, mAb2, mAb3, mAb4 and mAb5. Typically, methods of cell-free expression of proteins or antibodies are already described (Stech et al., 2017, Sci. Rep. 7, 12030).

### Evaluating Blood Baseline Vγ9V52 T Cell Counts or Vγ9+ T Cell Density

The methods of the present disclosure enable to predict the level of response of a subject to an activating anti-BTN3A antibody treatment as described above. In certain embodiments, the prediction is based on the evaluation of the blood baseline Vγ9Vδ2 T cell counts. In other embodiments, the prediction is based on the evaluation of Vγ9+ T cell density from a biopsy of the tumor of the patient. In other embodiments, the prediction is based on both the evaluation of the blood baseline Vγ9Vδ2 T cell counts and Vγ9+ T cell density.

As used herein, the term “predict” refers to a method that allows determining with a certain level of probability (statistically significant), prior to treatment, the level of response to said treatment. Accordingly, the term “predict” does not necessarily consist of an absolute response. Rather, it may consist of a response allowing to determine a higher probability of the patient to be a responder, as compared to the average probability in the patient population without the selection step.

As used herein, a “response to activating anti-BTN3A antibody treatment” or equally a “clinical response to activating anti-BTN3A antibody treatment” is observed when at least one of the symptoms of a cancer to be treated by said activating anti-BTN3A antibody treatment is decreased after treatment as compared to said symptom prior to the treatment.

As used herein, the term “decrease” or “increase” means a statistically significant decrease or increase of a control value, preferably, at least 10%, at least 20%, at least 30%, at least 40%, at least 50%, at least 90%, or at least 99% decrease or increase of the control value.

According to certain embodiments of the methods of the invention, the patient is predicted to be a responder or a non-responder based on evaluation of the expression of blood baseline Vγ9Vδ2 T cell counts as determined in a volume of a blood sample of said subject, prior to the treatment.

According to other embodiments of the methods of the invention, the patient is predicted to be responder or non-responder based on evaluation of the expression of Vγ9+ T cell density as determined in a volume of a tumor biopsy of said subject, prior to the treatment.

The term “responder” as used herein means a patient that demonstrates or is likely to demonstrate a better clinical response to activating anti-BTN3A treatment as compared to a non-responder subject. In an embodiment, a responder is a patient suffering from cancer who demonstrates or is likely to demonstrate significant size reduction of tumors, for example, as determined by standard tumor assessment, e.g. RECIST, iRECIST or RECIL for solid tumors, or other standard response evaluation for hematologic indications, e.g. Cheson/IWG.

In certain methods of the present disclosure, said subject has been selected for a treatment with said anti-BTN3A antibody by evaluating the blood baseline Vγ9Vδ2 T cell counts in said subject. The higher the blood baseline Vγ9Vδ2 T cell counts is, the better is the response to said anti-BTN3A activating antibody treatment. In other methods of the present disclosure, said subject has been selected for a treatment with said anti-BTN3A antibody by evaluating the tumor Vγ9+ T cell density from a biopsy of the tumor of said subject. The higher the tumor baseline Vγ9+ T cell density is, the better is the response to said anti-BTN3A activating antibody treatment.

The inventors have indeed provided clinical evidence of use of blood basal γδ T cell counts or tumor Vγ9+ T cell density as a predictive marker for selecting responders for activating anti-BTN3A antibodies, typically mAb1 activating anti-BTN3A antibody.

### Methods Comprising Determining the Baseline Vγ9Vδ2 T Cell Counts in a Blood Sample

In specific embodiments, the methods of the disclosure comprise the step of determining the baseline Vγ9Vδ2 T cell counts in a blood sample obtained from said subject to be treated with an activating anti-BTN3A antibody treatment.

As used herein, the blood sample is obtained by standard blood collection methods such as vacutainer.

Determining the number of γδ T cells in a blood sample may be achieved by any routine techniques of immunophenotyping. Typically, routine/standard flow cytometry method using beads for calibration. Appropriate conditions to the particular assay and components thereof will be well known to one skilled in the art.

This assessment of the baseline Vγ9Vδ2 T cell counts is typically made less than 4 days, preferably, less than 48 hours, more preferably less than 24 hours prior to first administration of said activating anti-BTN3A antibody.

It is anticipated that a high baseline Vγ9Vδ2 T cell counts is predictive of a better response. The exact baseline Vγ9Vδ2 T cell counts to be used for selecting a patient for eligibility to the treatment may however be determined by the skilled person, in particular depending on the clinical parameters of the patient (e.g. age, sex, prior lines of treatment), cancer diagnostics, and the protocol used (in particular if said activating anti-BTN3A antibody is used as monotherapy or in combination with other therapies, typically anti-PD1 or anti-PDL1 therapy).

In specific embodiments, said subject is selected for activating anti-BTN3A treatment if the baseline circulating Vγ9Vδ2 T cell counts is higher than 1000 cells/mL, 2000 cells/mL, 3000 cells/mL, 4000 cells/mL, 5000 cells/mL, 6000 cells/mL, 7000 cells/mL, 8000 cells/mL, 9000 cells/mL, 10 000 cells/mL, 11 000 cells/mL, 12 000 cells/mL, 13 000 cells/mL, 14 000 cells/mL, 15 000 cells/mL, 16 000 cells/mL, 17 000 cells/mL, 18 000 cells/mL, 19 000 cells/mL or higher than 20 000 cells/mL.

In specific embodiments, said subject is suffering from a cancer with solid tumors, and is selected for activating anti-BTN3A treatment (typically mAb1 treatment, preferably two or more injections at a dose comprised between 7 and 200 mg) if the baseline circulating Vγ9Vδ2 T cell counts is higher than 20 000 cells/mL in said subject.

In specific embodiments, said subject is suffering from ovarian cancer and is selected for activating anti-BTN3A treatment (typically mAb1 treatment, preferably two or more injections at a dose comprised between 7 and 200 mg) if the baseline circulating Vγ9Vδ2 T cell counts in said subject is higher than 20 000 cells/mL.

In specific embodiments, said subject is suffering from head and neck squamous cell cancer and is selected for activating anti-BTN3A treatment (typically mAb1 treatment, preferably two or more injections at a dose comprised between 7 and 200 mg) if the baseline circulating Vγ9Vδ2 T cell counts is higher than 20 000 cells/mL in said subject.

The disclosure also relates to a method for assessing the eligibility of a subject for an activating anti-BTN3A treatment in need thereof, said method comprising determining the baseline circulating Vγ9Vδ2 T cell counts in a blood sample of said subject, wherein a subject eligible for an activating anti-BTN3A treatment has a baseline Vγ9Vδ2 T cell counts above a certain reference value, typically set up between 1000 and 20 000 cells/mL.

### Methods Comprising Determining Vγ9+ T Cell Density in a Tumor Biopsy

In specific embodiments, the methods of the disclosure comprise the step of determining Vγ9+ T cell density in a tumor biopsy obtained from said subject to be treated with an activating anti-BTN3A antibody treatment.

Determining the Vγ9+ T cell density may be achieved by any routine techniques of immunophenotyping. Typically, Vγ9+ T cell density is assessed by immunohistochemistry in a tumor biopsy with a monoclonal antibody specifically directed to Vγ9+ TCR, such as the antibody 7B6, for example as described in the Examples.

This assessment of the baseline Vγ9+ T cell density is typically made less than 4 days, preferably, less than 48 hours, more preferably less than 24 hours prior to first administration of said activating anti-BTN3A antibody to said subject in need thereof.

It is anticipated that a high Vγ9+ T cell density is predictive of a better response. The exact Vγ9+ T cell density to be used for selecting a patient for eligibility to the treatment may however be determined by the skilled person, in particular depending on the clinical parameters of the patient (e.g. age, sex, prior lines of treatment), cancer diagnostics, and the protocol used (in particular if said activating anti-BTN3A antibody is used as monotherapy or in combination with other therapies, typically anti-PD1 or anti-PDL1 therapy).

In specific embodiments, said subject is selected for activating anti-BTN3A treatment if the baseline Vγ9+ T cell density is higher than 1 cell/mm², 2 cells/mm², 3 cells/mm², or 4 cells/mm² in the tumor biopsy from said subject.

In specific embodiments, said subject is suffering from a cancer with solid tumors, and is selected for activating anti-BTN3A treatment (typically mAb1 treatment, preferably two or more injections at a dose comprised between 7 and 200 mg) if the baseline Vγ9+ T cell density is higher than 3 cells/mm² or higher than 4 cells/mm² in the tumor biopsy from said subject.

In specific embodiments, said subject is suffering from ovarian cancer and is selected for activating anti-BTN3A treatment (typically mAb1 treatment, preferably two or more injections at a dose comprised between 7 and 200 mg) if the baseline Vγ9+ T cell density is higher than 3 cells/mm² or higher than 4 cells/mm² in the tumor biopsy from said subject.

In specific embodiments, said subject is suffering from head and neck squamous cell cancer and is selected for activating anti-BTN3A treatment (typically mAb1 treatment, preferably two or more injections at a dose comprised between 7 and 200 mg) if the baseline Vγ9+ T cell density is higher than 3 cells/mm² or higher than 4 cells/mm² in the tumor biopsy from said subject.

The disclosure also relates to a method for assessing the eligibility of a subject for an activating anti-BTN3A treatment in need thereof, said method comprising determining the baseline Vγ9+ T cell density in a tumor biopsy of said subject, wherein a subject eligible for an activating anti-BTN3A treatment has a baseline Vγ9+ T cell density which is higher than 3 cells/mm² or higher than 4 cells/mm² as determined in the tumor biopsy from said subject.

### Specific Embodiments of the Methods of the Disclosure

E1. An isolated activating anti-BTN3A antibody which induces the activation of Vγ9Vδ2 T cells, for use in treating a tumor in a human subject in need thereof, wherein said subject has been selected for said treatment with said anti-BTN3A antibody by either (i) evaluating the blood baseline Vγ9Vδ2 T cell counts in a blood sample of said subject prior to the treatment or (ii) the baseline Vγ9+ T cell density in a tumor biopsy of said subject prior to the treatment.

E2. The activating anti-BTN3A antibody for use according to E1, wherein said subject has been selected for said treatment when either (i) the baseline circulating Vγ9Vδ2 T cell counts is higher than 5000 cells/mL, higher than 10 000 cells/mL, or higher than 20 000 cells/mL, as determined in the blood sample or (ii) the baseline Vγ9+ T cell density is higher than 2 cells/mm², or higher than 3 cells/mm² or higher than 4 cells/mm², as determined in the tumor biopsy.

E3. The activating anti-BTN3A antibody for use according to E1 or E2, wherein said subject is suffering from a non-hematological cancer.

E4. The activating anti-BTN3A antibody for use according to E3, wherein said subject is suffering from a cancer selected from melanoma, pancreatic ductal adenocarcinoma (PDAC), colorectal cancer, ovarian cancer, breast cancer, gastric cancer, bladder, prostate, lung cancer such as non-small cell lung cancer (NSCLC), head and neck squamous cell cancer and urothelial cancer,

E5. The activating anti-BTN3A antibody for use according to E4, wherein said subject is suffering from head and neck squamous cell cancer (HNSCC), or ovarian cancer.

E6. The activating anti-BTN3A antibody for use according to any one of E1-E5, wherein said subject is having relapsed/refractory solid tumors.

E7. The activating anti-BTN3A antibody for use according to E1 or E2, wherein said subject is suffering from a hematological malignancy.

E8. The activating anti-BTN3A antibody for use according to E7, wherein said subject is suffering from a hematological malignancy selected from B-cell lymphoid neoplasm, T-cell lymphoid neoplasm, non-Hodgkin lymphoma (NHL), B-NHL, diffuse large B cell lymphoma (DLBCL), T-NHL, chronic lymphocytic leukemia (CLL), small lymphocytic lymphoma (SLL), mantle cell lymphoma (MCL), NK-cell lymphoid neoplasm and myeloid cell lineage neoplasm including acute myeloid leukemia (AML).

E9. The activating anti-BTN3A antibody for use according to any one of E1-E8, wherein said activating anti-BTN3A antibody binds to human BTN3A polypeptide with a K_{D} of 10 nM or less, preferably with a K_{D} of 5 nM or less, e.g. between 50 μM and 5 nM, as measured by surface plasmon resonance (SPR).

E10. The activating anti-BTN3A antibody for use according to any one of E1-E9, wherein said activating anti-BTN3A antibody cross-reacts to cynomolgus BTN3A with a K_{D} of 100 nM or less, preferably with a K_{D} of 10 nM or less, as measured by SPR.

E11. The activating anti-BTN3A antibody for use according to any one of E1-E10, wherein, either, said activating anti-BTN3A antibody induces in vitro the activation of Vγ9Vδ2-T cells in human PBMC, with an EC₅₀ below 0.1 mg/mL, preferably of 0.01 mg/mL or below, e.g. between 100 μg/mL and 0.1 mg/mL, as measured by surface expression of the activation markers CD69; or, said activating anti-BTN3A antibody induces the activation of Vγ9Vδ2 T cells in co-culture with BTN3 expressing cells, with an EC₅₀ below 5 mg/mL, preferably of 1 mg/mL or below, e.g. between 100 ng/mL and 5 mg/mL, as measured in a degranulation assay.

E12. The activating anti-BTN3A antibody for use according to any one of E1-E11, wherein said activating anti-BTN3A antibody comprises HCDR1 of SEQ ID NO:12, HCDR2 of SEQ ID NO:13, HCDR3 of SEQ ID NO:14, LCDR1 of SEQ ID NO:15, LCDR2 of SEQ ID NO:16 and LCDR3 of SEQ ID NO:17.

E13. The activating anti-BTN3A antibody for use according to any one of E1-E12, wherein said activating anti-BTN3A antibody is a humanized antibody.

E14. The activating anti-BTN3A antibody for use according to any one of E1-E13, wherein said activating anti-BTN3A antibody includes at least the following amino acid mutations in the VH framework regions: VSQ; V11L; K12V; R66K; S74F; 175S; E81Q; S82AR; R82BS; R83T; D85E; T87S; L108S; and at least the following amino acid mutations in the Vk framework regions: TSN; V15L; R18T; V19I; K42N; A43I; D70G; F73L; Q100G.

E15. The activating anti-BTN3A antibody for use according to any one of E1-E14, wherein said activating anti-BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E16. The activating anti-BTN3A antibody for use according to any one of E1-E15, wherein said activating anti-BTN3A antibody comprises a variable heavy chain (VH) of SEQ ID NO:1 and a variable light chain (VL) of SEQ ID NO:2.

E17. The activating anti-BTN3A antibody for use according to any one of E1-E15, wherein said activating anti-BTN3A antibody comprises a variable heavy chain (VH) of SEQ ID NO:1 and a variable light chain (VL) of SEQ ID NO:3.

E18. The activating anti-BTN3A antibody for use according to any one of E1-E17, wherein said activating anti-BTN3A antibody comprises a silent Fc region, typically a mutant IgG1 constant region or mutant IgG4 constant region.

E19. The activating anti-BTN3A antibody for use according to E18, wherein said mutant IgG1 constant region is IgG1 triple mutant L247F L248E and P350S.

E20. The activating anti-BTN3A antibody for use according to E18, wherein said mutant IgG4 constant region is IgG4 double mutant S241P L248E.

E21. The activating anti-BTN3A antibody for use according to any one of E1-E17, wherein said activating anti-BTN3A antibody comprises a heavy chain of SEQ ID NO:4 and a light chain of SEQ ID NO:6.

E22. The activating anti-BTN3A antibody for use according to any one of E1-E17, wherein said activating anti-BTN3A antibody comprises a heavy chain of SEQ ID NO:4 and a light chain of SEQ ID NO:7.

E23. The activating anti-BTN3A antibody for use according to any one of E1-E17, wherein said activating anti-BTN3A antibody comprises a heavy chain of SEQ ID NO:5 and a light chain of SEQ ID NO:6.

E24. The activating anti-BTN3A antibody for use according to any one of E1-E17, wherein said activating anti-BTN3A antibody comprises a heavy chain of SEQ ID NO:5 and a light chain of SEQ ID NO:7.

E25. The activating anti-BTN3A antibody for use according to any one of E1-E24, wherein said activating anti-BTN3A antibody is administered in combination with a cytokine

E26. The activating anti-BTN3A antibody for use according to any one of E25, wherein said cytokine is an IL2 or IL15 agonist.

E27. The activating anti-BTN3A antibody for use according to any one of E1-E26, wherein said anti-BTN3A antibody is administered in combination with an immunotherapeutic agent.

E28. The activating anti-BTN3A antibody for use according to E27, wherein said immunotherapeutic agent is an anti-PD1 or anti-PD-L1 antibody.

E29. The activating anti-BTN3A antibody for use according to E28, wherein said anti-PD1 or anti-PD-L1 antibody is selected from nivolumab, pembrolizumab, avelumab, durvalumab, cemiplimab, or atezolizumab, preferably pembrolizumab.

E30. The activating anti-BTN3A antibody for use according to E27, wherein said anti-BTN3A antibody is administered in combination with (i) an anti-PD1 or anti-PD-L1 antibody and (ii) a cytokine such IL2 or IL15 agonist or their derivatives, pegylated variants and superagonists variants thereof.

E31. The activating anti-BTN3A antibody for use according to any one of E1-E30, wherein the therapeutic dose of the activating anti-BTN3A antibody is within 7 and 200 mg per administration.

E32. The activating anti-BTN3A antibody for use according to any one of E1-E31, wherein said anti-BTN3A antibody is mAb1 and the therapeutic dose of the activating anti-BTN3A antibody is within 7 and 200 mg per administration.

E33. The activating anti-BTN3A antibody for use according to any one of E1-E32, wherein said activating anti-BTN3A antibody is administered intravenously at least twice at a dose comprised between 7 and 200 mg each dose, preferably the second dose being administered at least 15 days after the first dose, typically after about 21 days.

E34. The activating anti-BTN3A antibody for use according to any one of E1-E33, wherein said activating anti-BTN3A antibody is administered at a dose selected from 7, 10, 20, 50, 75, 100, 125, 150, 170 or 200 mg.

E35. The activating anti-BTN3A antibody for use according to any one of E1-E34, wherein said activating anti-BTN3A antibody is mAb1 and is administered at a dose selected from 7, 10, 20, 50, 75, 100, 125, 150, 170 or 200 mg.

E36. The activating anti-BTN3A antibody for use according to any one of E1-E34, wherein said activating anti-BTN3A antibody is mAb1 and is administered at a dose selected from 7, 10, 20, 50, 75, 100, 125, 150, 170 or 200 mg, in combination with anti-PD1 or anti-PD-L1 antibody, e.g. Pembrolizumab.

E37. The activating anti-BTN3A antibody for use according to any one of E1-E34, wherein said activating anti-BTN3A antibody is mAb1 and said subject is suffering from head and neck squamous cell cancer (HNSCC), or ovarian cancer, and said subject has been selected for said treatment when either, (i) the baseline circulating Vγ9Vδ2 T cell counts is higher than 5000 cells/mL, higher than 10 000 cells/mL, or higher than 20 000 cells/mL as determined in a blood sample, or (ii) the baseline Vγ9+ T cell density is higher than 2 cells/mm², or higher than 3 cells/mm² or higher than 4 cells/mm², as determined in the tumor biopsy.

E38. A method for treating a tumor in a human subject in need thereof, which comprises administering a therapeutically efficient amount of an activating anti-BTN3A antibody, wherein said human subject has been selected for said anti-BTN3A activating antibody treatment, by evaluating either (i) the blood baseline Vγ9Vδ2 T cell counts from a blood sample or (ii) the baseline Vγ9+ T cell density from a tumor biopsy of said subject.

E39. The method of E38, wherein said subject has been selected for said treatment when either (i) the baseline circulating Vγ9Vδ2 T cell counts is higher than 5000 cells/mL, higher than 10 000 cells/mL, or higher than 20 000 cells/mL or (ii) the baseline Vγ9+ T cell density is higher than 2 cells/mm², or higher than 3 cells/mm² or higher than 4 cells/mm², as determined in the tumor biopsy.

E40. The method of E38 or E39, wherein said subject is suffering from a non-hematological cancer.

E41. The method of E40, wherein in said subject is suffering from a cancer selected from melanoma, pancreatic ductal adenocarcinoma (PDAC), colorectal cancer, ovarian cancer, breast cancer, gastric cancer, bladder, prostate, lung cancer such as non-small cell lung cancer (NSCLC), head and neck squamous cell cancer and urothelial cancer,

E42. The method of E40, wherein said subject is suffering from head and neck squamous cell cancer (HNSCC), or ovarian cancer.

E43. The method of any one of E38-E42, wherein said subject is having relapsed/refractory solid tumors.

E44. The method of any one of E38-E43, wherein said subject is a subject is suffering from a hematological malignancy.

E45. The method of E44, wherein said subject is suffering from a hematological malignancy selected from B-cell lymphoid neoplasm, T-cell lymphoid neoplasm, non-Hodgkin lymphoma (NHL), B-NHL, diffuse large B cell lymphoma (DLBCL), T-NHL, chronic lymphocytic leukemia (CLL), small lymphocytic lymphoma (SLL), mantle cell lymphoma (MCL), NK-cell lymphoid neoplasm and myeloid cell lineage neoplasm including acute myeloid leukemia (AML).

E46. The method of any one of E38-E45, wherein said activating anti-BTN3A antibody binds to human BTN3A polypeptide with a K_{D} of 10 nM or less, preferably with a K_{D} of 5 nM or less, e.g. between 50 μM and 5 nM, as measured by surface plasmon resonance (SPR).

E47. The method of any one of E38-E46, wherein said activating anti-BTN3A antibody cross-reacts to cynomolgus BTN3A with a K_{D} of 100 nM or less, preferably with a K_{D} of 10 nM or less, as measured by SPR.

E48. The method of any one of E38-E47, wherein said activating anti-BTN3A antibody induces in vitro the activation of Vγ9Vδ2 T cells in human PBMC, with an EC₅₀ below 0.1 mg/mL, preferably of 0.01 mg/mL or below, e.g. between 100 μg/mL and 0.1 mg/mL, as measured by surface expression of the activation marker CD69.

E49. The method of any one of E38-E48, wherein said activating anti-BTN3A antibody induces the activation of Vγ9Vδ2 T cells in co-culture with BTN3 expressing cells, with an EC₅₀ below 5 mg/mL, preferably of 1 mg/mL or below, e.g. between 100 ng/mL and 5 mg/mL, as measured in a degranulation assay.

E50. The method of any one of E38-E49, wherein said activating anti-BTN3A antibody comprises HCDR1 of SEQ ID NO:12, HCDR2 of SEQ ID NO:13, HCDR3 of SEQ ID NO:14, LCDR1 of SEQ ID NO:15, LCDR2 of SEQ ID NO:16 and LCDR3 of SEQ ID NO:17.

E51. The method of any one of E38-E50, wherein said activating anti-BTN3A antibody is a humanized antibody.

E52. The method of E51, wherein said activating anti-BTN3A antibody includes at least the following amino acid mutations in the VH framework regions: VSQ; V11L; K12V; R66K; S74F; 175S; E81Q; S82AR; R82BS; R83T; D85E; T87S; L108S; and at least the following amino acid mutations in the Vk framework regions: TSN; V15L; R18T; V19I; K42N; A43I; D70G; F73L; Q100G.

E53. The method of any one of E38-E51, wherein said activating anti-BTN3A antibody comprises a mutant or chemically modified IgG1 constant region, wherein said mutant or chemically modified IgG1 constant region confers no or decreased binding to Fcg receptors when compared to a corresponding antibody with wild type IgG1 isotype constant region.

E54. The method of any one of E38-E53, wherein said activating anti-BTN3A antibody comprises a variable heavy chain (VH) of SEQ ID NO:1 and a variable light chain (VL) of SEQ ID NO:2.

E55. The method of any one of E38-E53, wherein said activating anti-BTN3A antibody comprises a variable heavy chain (VH) of SEQ ID NO:1 and a variable light chain (VL) of SEQ ID NO:3.

E56. The method of any one of E38-E55, wherein said activating anti-BTN3A antibody comprises a silent Fc region, typically a mutant IgG1 constant region or mutant IgG4 constant region.

E57. The method of E56, wherein said mutant IgG1 constant region is IgG1 triple mutant L247F L248E and P350S.

E58. The method of E56, wherein said mutant IgG4 constant region is IgG4 double mutant S241P L248E.

E59. The method of any one of E38-E58, wherein said activating anti-BTN3A antibody comprises a heavy chain of SEQ ID NO:4 and a light chain of SEQ ID NO:6.

E60. The method of any one of E38-E58, wherein said activating anti-BTN3A antibody comprises a heavy chain of SEQ ID NO:4 and a light chain of SEQ ID NO:7.

E61. The method of any one of E38-E58, wherein said activating anti-BTN3A antibody comprises a heavy chain of SEQ ID NO:5 and a light chain of SEQ ID NO:6.

E62. The method of any one of E38-E58, wherein said activating anti-BTN3A antibody comprises a heavy chain of SEQ ID NO:5 and a light chain of SEQ ID NO:7.

E63. The method of any one of E38-E62, wherein said activating anti-BTN3A antibody is administered in combination with a cytokine

E64. The method of E63, wherein said cytokine is an IL2 or IL15 agonist.

E65. The method of any one of E38-E64, wherein said activating anti-BTN3A antibody is administered in combination with an immunotherapeutic agent.

E66. The method of E65, wherein said immunotherapeutic agent is an anti-PD1 or anti-PD-L1 antibody.

E67. The method of E66, wherein said anti-PD1 or anti-PD-L1 antibody is selected from nivolumab, pembrolizumab, avelumab, durvalumab, cemiplimab, or atezolizumab, preferably pembrolizumab.

E68. The method of E66, wherein said activating anti-BTN3A antibody is administered in combination with (i) an anti-PD1 or anti-PD-L1 antibody and (ii) a cytokine such IL2 or IL15 agonist or their derivatives, pegylated variants and superagonists variants thereof.

E69. The method of any one of E38-E68, wherein a therapeutic dose of the activating anti-BTN3A antibody is within 7 and 200 mg per administration.

E70. The method of E69, wherein said anti-BTN3A antibody is mAb1 and the therapeutic dose of the activating anti-BTN3A antibody is within 7 and 200 mg per administration.

E71. The method of any one of E38-E70, wherein said activating anti-BTN3A antibody is administered intravenously at least twice at a dose comprised between 7 and 200 mg each dose, typically the second dose being administered at least 15 days after the first dose, for example, 21 days after the first dose.

E72. The method of any one of E38-E71, wherein said activating anti-BTN3A antibody is administered at a dose selected from 7, 10, 20, 50, 75, 100, 125, 150, 170 or 200 mg.

E73. The method of any one of E38-E72, wherein said activating anti-BTN3A antibody is mAb1 which is administered intravenously at least twice at a dose comprised between 7 and 200 mg each dose, preferably the second dose being administered at least 15 days after the first dose, typically after about 21 days.

E74. The method of any one of E38-E73, wherein said activating anti-BTN3A antibody is mAb1 and is administered at a dose selected from 7, 10, 20, 50, 75, 100, 125, 150, 170 or 200 mg.

E75. The method of any one of E38-E74, wherein said activating anti-BTN3A antibody is mAb1 and is administered at a dose selected from 7, 10, 20, 50, 75, 100, 125, 150, 170 or 200 mg, in combination with anti-PD1 or anti-PD-L1 antibody, e.g. Pembrolizumab.

E76. The method of any one of E38-E75, wherein said activating anti-BTN3A antibody is mAb1 and said subject is suffering from head and neck squamous cell cancer (HNSCC), or ovarian cancer, and said subject has been selected for said treatment when the baseline circulating Vγ9Vδ2 T cell counts is higher than 5000 cells/mL, higher than 10 000 cells/mL, or higher than 20 000 cells/mL.

E77. Use of an activating anti-BTN3A antibody as herein disclosed, in the manufacture of a drug for treating a subject in need thereof, wherein said subject has been selected for said treatment by evaluating either (i) the blood baseline Vγ9Vδ2 T cell counts in a blood sample of said subject, as herein described throughout the specification; or (ii) the baseline Vγ9+ T cell density is higher than 2 cells/mm², or higher than 3 cells/mm² or higher than 4 cells/mm², as determined in the tumor biopsy of said subject, as herein described throughout the specification.

E78. A method for determining eligibility to a treatment with an activating anti-BTN3A antibody as herein disclosed, in a subject in need thereof, said method comprising evaluating either (i) the blood baseline Vγ9Vδ2 T cell counts in a blood sample of said subject, as herein described throughout the specification; or (ii) the baseline Vγ9+ T cell density is higher than 2 cells/mm², or higher than 3 cells/mm² or higher than 4 cells/mm², as determined in the tumor biopsy of said subject, as herein described throughout the specification.

E79. An isolated activating anti-BTN3A antibody which induces the activation of Vγ9Vδ2 T cells, for use in treating a tumor in a human subject in need thereof, wherein said subject is having relapsed or refractory tumors after anti-PD1 or anti-PD-L1 treatment, and said subject is administered a therapeutically efficient amount of an anti-PD1 or anti-PD-L1 agent in combination with a therapeutically efficient amount of said activating anti-BTN3A antibody.

E80. An isolated activating anti-BTN3A antibody which induces the activation of Vγ9Vδ2 T cells, for use in treating a tumor in a human subject in need thereof, wherein said tumor is a solid tumor, in particular selected from the group consisting of bladder cancer, melanoma, non small cell lung cancer, and head and neck squamous cell carcinoma.

E81. The activating anti-BTN3A antibody which induces the activation of Vγ9Vδ2 T cells, for use in treating hematological malignancy, wherein said hematological malignancy is selected from diffuse large B cell lymphoma and acute myeloid leukemia.

E82. The activating anti-BTN3A antibody for use according to E79, for use in treating a tumor in a human subject in need thereof, wherein said tumor is a solid tumor, in particular selected from the group consisting of bladder cancer, melanoma, non small cell lung cancer, and head and neck squamous cell carcinoma.

E83. The activating anti-BTN3A antibody for use according to any one of E79-E82, wherein said activating anti-BTN3A antibody binds to human BTN3A polypeptide with a K_{D} of 10 nM or less, preferably with a K_{D} of 5 nM or less, e.g. between 50 μM and 5 nM, as measured by surface plasmon resonance (SPR).

E84. The activating anti-BTN3A antibody for use according to any one of E79-E83, wherein said activating anti-BTN3A antibody cross-reacts to cynomolgus BTN3A with a K_{D} of 100 nM or less, preferably with a K_{D} of 10 nM or less, as measured by SPR.

E85. The activating anti-BTN3A antibody for use according to any one of E79-E84, wherein said activating anti-BTN3A antibody induces in vitro the activation of Vγ9Vδ2-T cells in human PBMC, with an EC₅₀ below 0.1 mg/mL, preferably of 0.01 mg/mL or below, e.g. between 100 μg/mL and 0.1 mg/mL, as measured by surface expression of the activation markers CD69.

E86. The activating anti-BTN3A antibody for use according to any one of E79-E85, wherein said antibody induces the activation of Vγ9Vδ2 T cells in co-culture with BTN3 expressing cells, with an EC₅₀ below 5 mg/mL, preferably of 1 mg/mL or below, e.g. between 100 ng/mL and 5 mg/mL, as measured in a degranulation assay.

E87. The activating anti-BTN3A antibody for use according to any one of E79-E86, wherein said activating anti-BTN3A antibody comprises HCDR1 of SEQ ID NO:12, HCDR2 of SEQ ID NO:13, HCDR3 of SEQ ID NO:14, LCDR1 of SEQ ID NO:15, LCDR2 of SEQ ID NO:16 and LCDR3 of SEQ ID NO:17.

E88. The activating anti-BTN3A antibody for use according to any one of E79-E87, wherein said activating anti-BTN3A antibody is a humanized antibody.

E89. The activating anti-BTN3A antibody for use according to any one of E79-E88, wherein said activating anti-BTN3A antibody includes at least the following amino acid mutations in the VH framework regions: VSQ; V11L; K12V; R66K; S74F; 175S; E81Q; S82AR; R82BS; R83T; D85E; T87S; L108S; and at least the following amino acid mutations in the Vk framework regions: TSN; V15L; R18T; V19I; K42N; A43I; D70G; F73L; Q100G.

E90. The activating anti-BTN3A antibody for use according to any one of E79-E89, wherein said activating anti-BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E91. The activating anti-BTN3A antibody for use according to any one of E79-E90, wherein said activating anti-BTN3A antibody comprises a variable heavy chain (VH) of SEQ ID NO:1 and a variable light chain (VL) of SEQ ID NO:2.

E92. The activating anti-BTN3A antibody for use according to any one of E79-E90, wherein said activating anti-BTN3A antibody comprises a variable heavy chain (VH) of SEQ ID NO:1 and a variable light chain (VL) of SEQ ID NO:3.

E93. The activating anti-BTN3A antibody for use according to any one of E79-E92, wherein said activating anti-BTN3A antibody comprises a silent Fc region, typically a mutant IgG1 constant region or mutant IgG4 constant region.

E94. The activating anti-BTN3A antibody for use according to E93, wherein said mutant IgG1 constant region is IgG1 triple mutant L247F L248E and P350S.

E95. The activating anti-BTN3A antibody for use according to any one of E79-E94, wherein said activating anti-BTN3A antibody comprises a heavy chain of SEQ ID NO:4 and a light chain of SEQ ID NO:6.

E96. The activating anti-BTN3A antibody for use according to any one of E79-E95, wherein said activating anti-BTN3A antibody comprises a heavy chain of SEQ ID NO:4 and a light chain of SEQ ID NO:7.

E97. The activating anti-BTN3A antibody for use according to any one of E79-E96, wherein said activating anti-BTN3A antibody comprises a heavy chain of SEQ ID NO:5 and a light chain of SEQ ID NO:6.

E98. The activating anti-BTN3A antibody for use according to any one of E79-E97, wherein said activating anti-BTN3A antibody comprises a heavy chain of SEQ ID NO:5 and a light chain of SEQ ID NO:7.

E99. The activating anti-BTN3A antibody for use according to any one of E79-E98, wherein said activating anti-BTN3A antibody is administered in combination with a cytokine

E100. The activating anti-BTN3A antibody for use according to E99, wherein said cytokine is an IL2 or IL15 agonist.

E101. The activating anti-BTN3A antibody for use according to any one of E80-E100, wherein said anti-BTN3A antibody is administered in combination with an immunotherapeutic agent.

E102. The activating anti-BTN3A antibody for use according to E79-E101, wherein said anti-BTN3A antibody is administered in combination with an immunotherapeutic agent selected from the group consisting of anti-PD1 or anti-PD-L1 antibody.

E103. The activating anti-BTN3A antibody for use according to E79-E102, wherein said anti-BTN3A antibody is administered in combination with anti-PD1 or anti-PD-L1 antibody selected from the group consisting of nivolumab, pembrolizumab, avelumab, durvalumab, cemiplimab, or atezolizumab, preferably pembrolizumab.

E104. The activating anti-BTN3A antibody for use according to any one of E79-E103, wherein the therapeutic dose of the activating anti-BTN3A antibody is within 7 and 200 mg per administration, preferably within 20 and 75 mg.

E105. The activating anti-BTN3A antibody for use according to E79, wherein said anti-BTN3A antibody comprises a heavy chain of SEQ ID NO:4 and a light chain of SEQ ID NO:6, and the therapeutic dose of the activating anti-BTN3A antibody is within 7 and 200 mg per administration, preferably within 20 and 75 mg and wherein subject is administered with a therapeutically efficient amount of pembrolizumab.

E106. The activating anti-BTN3A antibody for use according to E80, wherein said anti-BTN3A antibody comprises a heavy chain of SEQ ID NO:4 and a light chain of SEQ ID NO:6, and the therapeutic unit dose of the activating anti-BTN3A antibody is within 7 and 200 mg per administration, preferably within 20 and 75 mg.

E107. The activating anti-BTN3A antibody for use according to any one of E79-E106, wherein said activating anti-BTN3A antibody is administered intravenously at least twice at a dose comprised between 7 and 200 mg each dose, preferably the second dose being administered at least 15 days after the first dose, typically after about 21 days.

E108. The activating anti-BTN3A antibody for use according to any one of E79-E107, wherein said activating anti-BTN3A antibody is administered at a dose selected from 7, 10, 20, 50, 75, 100, 125, 150, 170 or 200 mg.

E109. A method for treating a tumor in a human subject in need thereof, which comprises administering a therapeutically efficient amount of an activating anti-BTN3A which induces the activation of Vγ9Vδ2 T cells, in combination with a therapeutically efficient amount of an anti-PD1/PDL1 treatment, wherein said subject is having relapsed or refractory tumors to anti-PD1/PDL1 treatment.

E110. A method for treating a tumor in a human subject in need thereof, which comprises administering a therapeutically efficient amount of an activating anti-BTN3A which induces the activation of Vγ9Vδ2 T cells, wherein said tumor is a solid tumor, in particular selected from the group consisting of bladder cancer, melanoma, non small cell lung cancer, and head and neck squamous cell carcinoma.

E111. A method for treating a hematological malignancy in a human subject in need thereof, said method comprising administering a therapeutically efficient amount of an activating anti-BTN3A which induces the activation of Vγ9Vδ2 T cells, wherein said hematological malignancy is selected from diffuse large B cell lymphoma and acute myeloid leukemia.

E112. The method of E109, wherein said tumor is a solid tumor, in particular selected from the group consisting of bladder cancer, melanoma, non small cell lung cancer, and brain metastasis.

E113. The method of any one of E109-E112, wherein said activating anti-BTN3A antibody binds to human BTN3A polypeptide with a K_{D} of 10 nM or less, preferably with a K_{D} of 5 nM or less, e.g. between 50 μM and 5 nM, as measured by surface plasmon resonance (SPR).

E114. The method of any one of E109-E113, wherein said activating anti-BTN3A antibody cross-reacts to cynomolgus BTN3A with a K_{D} of 100 nM or less, preferably with a K_{D} of 10 nM or less, as measured by SPR.

E115. The method of any one of E109-E114, wherein said activating anti-BTN3A antibody induces in vitro the activation of Vγ9Vδ2 T cells in human PBMC, with an EC₅₀ below 0.1 mg/mL, preferably of 0.01 mg/mL or below, e.g. between 100 μg/mL and 0.1 mg/mL, as measured by surface expression of the activation markers CD69.

E116. The method of any one of E109-E115, wherein said antibody induces the activation of Vγ9Vδ2 T cells in co-culture with BTN3 expressing cells, with an EC₅₀ below 5 mg/mL, preferably of 1 mg/mL or below, e.g. between 100 ng/mL and 5 mg/mL, as measured in a degranulation assay.

E117. The method of any one of E109-E116, wherein said activating anti-BTN3A antibody comprises HCDR1 of SEQ ID NO:12, HCDR2 of SEQ ID NO:13, HCDR3 of SEQ ID NO:14, LCDR1 of SEQ ID NO:15, LCDR2 of SEQ ID NO:16 and LCDR3 of SEQ ID NO:17.

E118. The method of any one of E109-E117, wherein said activating anti-BTN3A antibody is a humanized antibody.

E119. The method of any one of E109-E118, wherein said activating anti-BTN3A antibody includes at least the following amino acid mutations in the VH framework regions: VSQ; V11 L; K12V; R66K; S74F; 175S; E81Q; S82AR; R82BS; R83T; D85E; T87S; L108S; and at least the following amino acid mutations in the Vk framework regions: TSN; V15L; R18T; V19I; K42N; A43I; D70G; F73L; Q100G.

E120. The method of any one of E109-E119, wherein said activating anti-BTN3A antibody comprises a mutant or chemically modified IgG1 constant region, wherein said mutant or chemically modified IgG1 constant region confers no or decreased binding to Fcg receptors when compared to a corresponding antibody with wild type IgG1 isotype constant region.

E121. The method of any one of E109-E120, wherein said activating anti-BTN3A antibody comprises a variable heavy chain (VH) of SEQ ID NO:1 and a variable light chain (VL) of SEQ ID NO:2.

E122. The method of any one of E109-E120, wherein said activating anti-BTN3A antibody comprises a variable heavy chain (VH) of SEQ ID NO:1 and a variable light chain (VL) of SEQ ID NO:3.

E123. The method of any one of E109-E122, wherein said activating anti-BTN3A antibody comprises a silent Fc region, typically a mutant IgG1 constant region or mutant IgG4 constant region.

E124. The method of any one of E109-E123, wherein said mutant IgG1 constant region is IgG1 triple mutant L247F L248E and P350S.

E125. The method of any one of E109-E124, wherein said activating anti-BTN3A antibody comprises a heavy chain of SEQ ID NO:4 and a light chain of SEQ ID NO:6.

E126. The method of any one of E109-E124, wherein said activating anti-BTN3A antibody comprises a heavy chain of SEQ ID NO:4 and a light chain of SEQ ID NO:7.

E127. The method of any one of E109-E124, wherein said activating anti-BTN3A antibody comprises a heavy chain of SEQ ID NO:5 and a light chain of SEQ ID NO:6.

E128. The method of any one of E109-E124, wherein said activating anti-BTN3A antibody comprises a heavy chain of SEQ ID NO:5 and a light chain of SEQ ID NO:7.

E129. The method of any one of E109-E128, wherein said activating anti-BTN3A antibody is administered in combination with a cytokine.

E130. The method of E129, wherein said cytokine is an IL2 or IL15 agonist.

E131. The method of any one of E109-E130, wherein said anti-BTN3A antibody is administered in combination with an immunotherapeutic agent.

E132. The method of any one of E109-E131, wherein said anti-BTN3A antibody is administered in combination with an immunotherapeutic agent selected from the group consisting of anti-PD1 or anti-PD-L1 antibody.

E133. The method of any one of E109-E132, wherein said anti-BTN3A antibody is administered in combination with anti-PD1 or anti-PD-L1 antibody selected from the group consisting of nivolumab, pembrolizumab, avelumab, durvalumab, cemiplimab, or atezolizumab, preferably pembrolizumab.

E134. The method of any one of E109-E133, wherein the therapeutic dose of the activating anti-BTN3A antibody is within 7 and 200 mg per administration, preferably within 20 and 75 mg.

E135. The method of any one of E109-E112, wherein said anti-BTN3A antibody comprises a heavy chain of SEQ ID NO:4 and a light chain of SEQ ID NO:6, and the therapeutic dose of the activating anti-BTN3A antibody is within 7 and 200 mg per administration, preferably within 20 and 75 mg and wherein subject is administered with a therapeutically efficient amount of pembrolizumab.

E136. The method of any one of E109-E112, wherein said anti-BTN3A antibody comprises a heavy chain of SEQ ID NO:4 and a light chain of SEQ ID NO:6, and the therapeutic dose of the activating anti-BTN3A antibody is within 7 and 200 mg per administration, preferably within 20 and 75 mg.

E137. The method of any one of E109-E136, wherein said activating anti-BTN3A antibody is administered intravenously at least twice at a dose comprised between 7 and 200 mg each dose, preferably the second dose being administered at least 15 days after the first dose, typically after about 21 days.

E138. The method of any one of E109-E137, wherein said activating anti-BTN3A antibody is administered at a dose selected from 7, 10, 20, 50, 75, 100, 125, 150, 170 or 200 mg.

E139. A method for enhancing immune cell infiltration in a tumor of a subject in need thereof, said method comprising administering an efficient amount of an activating anti-BTN3A antibody as disclosed herein, preferably in combination with an efficient amount of an anti-PD1 or anti-PDL1 agent, for example an anti-PD1 antibody, such as pembrolizumab.

E140. The method of E139, wherein said immune cells comprises Vγ9Vδ2 T cells and CD8+ T cells.

### Examples

### Methods to Characterize Anti-BTN3A Activating Antibodies for Use According to the Present Disclosure

### 1.1 Binding Affinity Assay: Multi-Cycle Kinetic Assay (SPR)

Multi-cycle kinetic analysis can be performed on anti-BTN3A antibodies using a Biacore T200 (serial no. 1909913) instrument running Biacore T200 Evaluation Software V2.0.1 (Uppsala, Sweden).

Purified antibodies are diluted to a concentration of 2 μg/ml in 2% BSA/PBS. At the start of each cycle, each antibody is captured on the Protein A at a density (RL) of ˜146.5 RU (theoretical value to obtain an RMax of ˜50 RU). Following capture, the surface is allowed to stabilize before injection of the BTN3A1 antigen (Sino Biological cat. no. 15973-H08H). BTN3A1 is titrated in 0.1% BSA/HBS-P+(running buffer) in a two-fold dilution range from 25 to 0.78 nM. The association phase is monitored for 400 seconds and the dissociation phase for 35 minutes (2100 seconds). Kinetic data is obtained using a flow rate of 50 μl/min to minimize any potential mass transfer effects. Regeneration of the Protein A surface is conducted using two injections of 10 mM glycine-HCL pH 1.5 at the end of each cycle. Two blanks (no BTN3A1) and a repeat of a single concentration of the analyte are performed for each tested antibody to check the stability of the surface and analyte over the kinetic cycles. The signal from the reference channel Fc1 is subtracted from that of Fc2, Fc3 and Fc4 to correct for differences in non-specific binding to a reference surface. Additionally, blank runs are subtracted for each Fc to correct any antigen-independent signal variation, such as drift. Sensorgrams are fitted using a one-to-one binding mathematical model with a global RMax parameter and no bulk signal (Constant RI=0 RU).

### 1.2 Binding Assay by Flow Cytometry on Human PBMCs

Anti-BTN3A antibodies for use according to the present disclosure may also be characterized for their binding to human PBMCs, isolated from blood of healthy donors. PBMCs are isolated from buffy coats using Lymphoprep (Axis-shield, Dundee, UK) density centrifugation. PBMCs are then frozen and stored at −80° C. or in liquid nitrogen until required.

100 μl cells at 1×10⁶ cells/ml are transferred to each well of a fresh U-shaped bottom 96-well plate, then the plate was centrifuged and supernatant discarded.

A serial dilution of the antibodies, 0.001 μg/ml to 150 μg/ml is prepared in PBS 2 mM EDTA. Human PBMCs were resuspended in 50 μl of the diluted test antibody titration series prepared.

After incubation for 30 minutes at 4° C. in the dark, the plate was centrifuged and washed twice with 150 μl/well of PBS 2 mM EDTA following which the wells are resuspended in 50 μl of a mix composed of goat anti-human antibody (PE labelled) diluted 1/100 and Live/dead neat IR diluted 1/500 in PBS 2 mM EDTA.

After incubation for 15 minutes at 4° C. in the dark, the plate is centrifuged and washed once with 150 μl/well PBS 2 mM EDTA following which the wells are resuspended in 200 μl PBS 2 mM EDTA. Cells are analyzed on a BD LSR Fortessa Cytometer. Data is analyzed using a FlowJo software (Version 10, FlowJo, LLC, Ashland, USA).

Same protocol may be performed on cynomolgus PBMCs and on Daudi Burkitt's lymphoma cell line.

### 1.3 In Vitro Functional Efficacy: γδ-T Cell Degranulation Assay

The assay consists of measuring activating or inhibitory effect of anti-BTN3A antibodies on γδ-T cell degranulation against Daudi Burkitt's lymphoma cell line (Harly et al., 2012). γδ-T cells are expanded from PBMCs of healthy donors by culturing with zoledronic acid (1 μM) and IL2 (200 Ui/ml) for 11-13 days. IL2 is added at day 5, day 8 and every 2 days thereafter. The percentage of γδ-T cells is determined at the initiation of culture and assessed for the time of culture by flow cytometry until it reached at least 80%. Frozen or fresh γδ-T cells are then used in degranulation assays against Daudi cell line (E:T ratio of 1:1), whereby the cells are co-cultured for 4 hours at 37° C. in presence of 10 μg/ml of the 7.2 and 20.1 humanized variants and their chimeric versions. Activation by PMA (20 ng/ml) plus lonomycin (1 μg/ml) served as positive control for γδ-T cell degranulation, and medium alone as negative control. At the end of 4 hour co-incubation, cells are analyzed by flow cytometry to evaluate the percentage of γδ-T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

The same protocol may be performed using AML blasts isolated from patients as target cells, in place of Daudi cells.

### 1.4 Determining Absolute Blood Baseline γδ T Cell Counts by Flow Cytometry

For assessment of γδ T cell absolute counting, fresh blood samples (100 μI) are mixed with a cocktail of antibodies (50 μI) prepared in PBS 1% foetal bovine serum (FBS), Calibration beads (for instance Trucount beads from BD) and a viability marker. Samples were incubated for 20 minutes at room temperature in the dark. Red blood cells (RBC) were lysed by adding 2 mL of pre-warmed 1×RBC lysis buffer (Biolegend #420302), vortexing and adding a further 2 mL of 1×RBC lysis buffer followed by incubating for 15 minutes at room temperature in the dark. After washes in PBS 1% FBS, the cell pellets were resuspended in 300 μL of PBS 1% FBS and acquired on a BD LSR Fortessa X-20 using a pre-defined application setting on BD FACS Diva software (v8.0). Data were normalized with the equation: ((MFI test-MFI IC)/(MFI test pre-dose-MFI IC pre-dose)×100). The analysis was performed using FlowJo Software (v10.6).

### 1.5 Determining Vγ9+ T Cell Density in a Tumor Biopsy

For assessment of Vγ9+ T cell density in a tumor biopsy, biopsies samples from human patients were collected pre-treatment and at day 28 post-treatment. At each time point, half of the biopsy was embedded in paraffin and the other half was snap-frozen in optimal cutting temperature compound (OCT). Immunohistochemistry was performed by Veracyte (Marseille, France) on Bond RX Automatic Stainer (Leica Biosystems) and images were acquired using a Nanozoomer XR scanner (Hamamatsu). Briefly, 6 μm thick sections of fresh frozen samples were fixed in zinc formalin for 5 min at RT, incubated 30 min with 5% human serum for blocking and stained with anti-panBTN3A mAb (clone 20.1) or anti-Vγ9TCR mAb (clone 7B6, Huang et al, Infection and Immunity 2008, pp 426-436) at 2 μg/mL for 45 minutes at RT. Antigen-antibody binding was revealed with Bond Polymer Refine RED Detection kit (Leica Biosystems). Tissue sections were then counter-stained with hematoxylin. Multiplex immunohistochemistry stainings for BTN3A2, BTN3A3, δTCR (clone H-41), CD3, CD4, FoxP3, Ki67, CD8, NKp46, and Granzyme B were performed using the Brightplex from Veracyte on a single FFPE slide. Briefly, 4 μm slides from paraffin embedded biopsies were dewaxed, treated for 20 min with Epitope Retrieval Solution I (Leica Biosystems) and successively stained with indicated mAbs. Antigen-antibody binding was revealed using MACH 2 HRP Polymer (Biocare). Tissue sections were then stained with ImmPACT™ AMEC Red (Vector Lab), and counter-stained with hematoxylin. Between each staining cycle, slides were AMEC destained by ethanol and antibodies were stripped with denaturing solution. Images were acquired between each cycle of staining.

### Example 1: Clinical Study—Summary

A First-In-Human, Two-Part, Open-Label, Clinical Study to Assess the Safety, Tolerability and Activity of Intravenous Doses of ICT01 as Monotherapy and in Combination with an Immune Checkpoint Inhibitor (ICI), in Patients with Advanced-Stage, Relapsed/Refractory Cancer (EVICTION Study)

Study Drug: ICT01; humanized activating anti-BTN3A immunoglobulin (Ig)G1 monoclonal antibody (mAb), engineered to reduce Fc-effector functions, targeting human butyrophilin-3A (BTN3A).

### Part 1 Objectives:

Primary: Characterize the overall safety and tolerability profile of a range of intravenous (IV) doses of ICT01 as monotherapy, and in combination with pembrolizumab in patients with advanced-stage, relapsed/refractory solid tumors or hematologic cancers.

### Secondary:

1. Characterize the pharmacokinetics (PK) and pharmacodynamics (PD) of IV ICT01 administered to patients with advanced-stage, relapsed/refractory solid tumors or hematologic cancers

2. Determine the recommended dose(s) for the expansion cohorts (Part 2) for ICT01 as monotherapy and in combination with pembrolizumab

3. Characterize the preliminary anti-tumor activity of a range of IV doses of ICT01 as monotherapy and in combination with pembrolizumab when administered to patients with advanced-stage, relapsed/refractory solid tumors or hematologic cancers

### Part 2 Objectives:

Primary; Characterize the preliminary anti-tumor activity of IV ICT01 as monotherapy and in combination with pembrolizumab in patients with advanced-stage, relapsed/refractory solid tumors or hematologic cancers.

### Secondary:

1. Characterize the overall safety and tolerability of IV ICT01 as monotherapy, and in combination with pembrolizumab in patients with advanced-stage, relapsed/refractory solid tumors or hematologic cancers.

2. Characterize the PK and PD of IV ICT01 administered to patients with advanced-stage, relapsed/refractory solid tumors or hematologic cancers.

### Study Design:

This is a phase I/11a, first-in-human, two-part, open-label study to characterize the safety, tolerability, PK, PD and anti-tumor activity of ICT01. Part 1 will be a dose escalation of IV ICT01 as monotherapy administered every 21 days to patients with advanced-stage, relapsed/refractory cancer (Group A: Mixed solid tumors; Group B: advanced hematologic malignancies). The combination of IV ICT01 with pembrolizumab (anti-PD-1; Keytruda®) will be evaluated in Group C (tumors that qualified for treatment with pembrolizumab but showed no response, progressed or relapsed during treatment).

Part 2 is the expansion stage of the study, where additional patients in two solid tumor indications (Group D & E) or a hematologic malignancy (Group F) will be treated with ICT01 doses identified in Part 1 that have demonstrated a favorable risk/benefit profile as monotherapy. Group G will be an expansion of the combination of ICT01 (up to 2 dose levels) with pembrolizumab in a single indication. The final specifics around doses and indications in Part 2 will be updated in the protocol via a substantial amendment before commencing this part of the study.

The route of administration of ICT01 will be IV infusion over 30 minutes.

Pembrolizumab (KEYTRUDA®) will be used for all patients in Group C at a dose of 200 mg IV q 21 days, which is the approved dose. According to the manufacturer, no dose reductions of KEYTRUDA are recommended. Withhold or discontinue KEYTRUDA to manage adverse reactions as described in APPENDIX: Pembrolizumab (Keytruda®) pembrolizumab (Keytruda®) Summary of Product Characteristics.

Group D (Ovarian Cancer) and E (Head and Neck Squamous Cell Cancer (HNSCC)): The 2 dose levels of ICT01 are 7 mg and 200 mg, which have both been shown to be pharmacodynamically active and safe. Each dose group will be up to 25 patients per indication. Patients will be randomly assigned in a 1:1 ratio to either 7 mg or 200 mg of ICT01 on Day 0 following the completion of all screening assessments and after confirmation of their eligibility.

Group F and G: A substantial amendment will be submitted prior to starting Part 2 in order to communicate the selected patient populations and the ICT01 dose(s) for each Group.

Patients will be evaluated for eligibility during the 3-week Screening Period. Biopsies will be collected as in Part 1. Patients will be treated every 21 days with IV ICT01 as monotherapy in Groups D, E, and F, or in combination with pembrolizumab in Group G.

### Study Population

### Inclusion Criteria for Part 1

The following criteria must be checked during the screening period and at baseline. ALL inclusion criteria must be met to include the subject in the study:
1) Male or female aged years
2) Voluntarily signed written informed consent before performance of any study-related screening procedures
3) Relapsed/refractory patients with histologically or cytologically confirmed diagnosis of advanced-stage or recurrent cancer including:
   a. Group A: bladder, breast, colorectal, gastric, melanoma, ovarian, prostate and PDAC
   b. Group B: hematologic malignancies including acute myeloid leukemia, acute lymphocytic leukemia, Diffuse large B cell lymphoma and follicular lymphoma
   c. Group C: melanoma, bladder, head and neck SCC, and non small cell lung cancer (approved indications in the US & EU for pembrolizumab)
4) Willingness to undergo baseline and on-study tumor biopsies
5) Eastern Cooperative Oncology Group (ECOG) performance status 1
6) Life expectancy >3 months as assessed by the Investigator
7) Clinical labs:
   a. Hematology:
      Hemoglobin ≥8.5 g/dL (equal to 5.28 mmol/L; transfusion dependent or independent);
      Group A/C only:
         platelet count ≥75×10⁹/L;
         lymphocyte count ≥0.5×10⁹/L;
         absolute neutrophil count ≥1.0×10⁹/L;
   b. Liver enzymes:
      aspartate transaminase (AST) and alanine transaminase (ALT) ≤2.5×upper limit of normal (ULN) (<5×ULN in the case of liver metastases);
      bilirubin ≤1.5×ULN (<2×ULN in case of liver metastases);
   c. Renal function: serum creatinine <1.5×ULN or creatinine clearance ≥50 mL/min (Cockcroft and Gault) for serum creatinine ≥1.5×ULN.
8) Contraceptives measures
   a. Women of childbearing potential must:
      i. have a negative pregnancy test within 1 week before first dose of study drug
      ii. use highly effective method(s) of birth control consistently and correctly during the study and for at least 5 months after the last dose of study drug
      iii. agree to not donate eggs (ova, oocytes) for the purposes of assisted reproduction during the study and for at least 5 months after the last dose of study
      iv. agree to no plan to breastfeed and no plan to become pregnant during the study and for at least 5 months after the last dose of study drug.
   b. Males who are sexually active must:
      i. agree to use a condom with spermicidal foam/gel/film/cream/suppository during the study and for at least 5 months after the last dose of study drug
      ii. agree to not donate sperm during the study and for at least 5 months after the last dose of study drug
      iii. no plan to father a child during the study or within 5 months after the last dose of study drug.
9) Women must not be breastfeeding
10) At least 1 measurable lesion per Response Evaluation Criteria in Solid Tumors (RECIST)/Response Evaluation Criteria in Lymphoma (RECIL) or >5% marrow blasts
11) Patients must have no available standard of care for their disease, as determined by the treating Investigator
12) Patients in the ICT01 Combination arms must meet the eligibility criteria in the approved package labeling of pembrolizumab and meet the following conditions:
   a. Must not be first-line patients (i.e., must meet Inclusion Criteria #11)
   b. Must not have any history of or ongoing interstitial lung disease
   c. Must not have undergone prior anterior organ transplantation, including allograft stem cell transplantation

### Part 2 Group D and E:

13) Replaces inclusion criterion #3: Relapsed/refractory patients with histologically or cytologically confirmed diagnosis of advanced-stage or recurrent cancer including:
   a. Group D: persistent or recurrent epithelial ovarian cancer, primary fallopian or primary peritoneal cancer; failed at least 1 prior systemic platinum-containing regimen
   b. Group E: metastatic or unresectable, recurrent HNSCC failed at least 1 prior systemic regimen
14) Replaces inclusion criterion #11: Patients must have received at least one line of treatment for their cancer prior to enrolling on the study
15) Circulating γ9δ2 T cell count ≥20000 cells/mL of blood during screening

### Statistical Analysis

### Analysis Populations

The safety population will consist of all patients who received at least 1 dose of ICT01 or ICT01 in combination with pembrolizumab, with solid tumors, hematologic tumors and combination therapy patients' data will be analyzed separately. The efficacy evaluable population will consist of all patients treated with at least 2 doses of (i) ICT01 (>1 month of therapy) or (ii) ICT01 in combination with pembrolizumab and without any protocol deviation likely to bias the efficacy evaluation, while an Intent to Treat (ITT) population will be used for exploratory purposes. For part 2, the efficacy evaluable population will consist of all treated patients with a week 8 anti-tumor assessment (e.g., RECIST).

### Primary Endpoints

Part 1. The primary endpoint of safety and tolerability will be evaluated in this study by the incidence, severity, and relationship of Treatment Emergent Adverse Events (TEAEs), Serious Adverse Events (SAEs), TEAEs leading to discontinuation of study treatment; and clinically significant findings on clinical laboratory tests, vital signs, ECGs, and physical examinations (Key secondary endpoint in Part 2).

Part 2. Disease Control Rate (DCR) that includes clinical response criteria as per RECIST, RECIL or as per disease-specific standards for the hematologic malignancies (e.g., Cheson/IWG Criteria for AML). The DCR is the sum of Complete Response/Remission (CR)+CR with incomplete recovery (CRi)+Partial Response/Remission (PR)+Stable Disease.

### Secondary Endpoints

In Part 1 the preliminary anti-tumor activity endpoints will be the DCR and Objective Response Rate (ORR) as per RECIST, RECIL or as per disease-specific standards in the hematologic indications (e.g., Cheson/IWG Criteria for AML). The immunotherapy Response Evaluation Criteria In Solid Tumors (iRECIST (will be considered exploratory).

In Part 2, Objective Response Rate (ORR) as per RECIST, RECIL or as per disease-specific standards for the hematologic malignancies (e.g., Cheson/IWG Criteria for AML). Objective response rate is the sum of Complete Response/Remission (CR)+CR with incomplete recovery (CRi)+Partial Response/Remission (PR). iRECIST will be exploratory.

Additional secondary endpoints include the safety and tolerability, time to progression (TTP) and progression-free survival (PFS).

In Parts 1 and 2, the PK parameters of ICT01 (including Cₘₐₓ, AUC, t_{1/2}, clearance) will be calculated by dose level. Likewise, the PD activity of ICT01 (by dose and patient population) will include the change from baseline in counts and activation status of γ9δ2 T cells and other immune cells in the peripheral blood, peripheral blood mononuclear cells (PBMCs) and tumor biopsies, circulating cytokine levels (including IFNγ, TNFα, IL-1β, IL-2, IL-4, IL-6, IL-8, IL-10, IL-17a and MCP-1), and expression of PD-L1, PD-1 and other immune cell markers in tumor biopsies and PBMCs.

Baseline BTN3A expression and γδ T cells from tumor biopsies, and baseline γδ T cells and BTN3A expression in the circulation will be characterized and used as covariates for the PD and clinical response analyses.

The primary PD activity measure to determine an active dose level of ICT01 will be a decrease from baseline and an increase in activation of circulating γδ T cells, as measured by flow cytometry.

### Example 2: Clinical Evidence of Use of γδ T Cell Counts or Vγ9+ T Cell Density as a Predictive Marker for Selecting Responders

### Materials and Methods

### Patients and Clinical Trial

EVICTION (NCT04243499) is a first-in-human, two-part, open-label, clinical study to assess the safety, tolerability, pharmacokinetics, pharmacodynamics and anti-tumor activity of IV doses of ICT01 (humanized antibody derived from murine mAb7.2) as monotherapy and in combination with pembrolizumab, in patients with advanced-stage, relapsed/refractory cancer. Adult patients must have failed all available standard of care for their cancer to be eligible, and not received other anti-cancer therapy during the trial, except patients in the combination arm of the trial. Patients receive ICT01 (Range: 20 μg to 200 mg) every 3 weeks with blood samples collected at multiple timepoints for safety measures, immunophenotyping on whole blood and serum cytokine analysis.

Tumor biopsies are collected at baseline and on day 28 and stained by immunohistochemistry for Vγ9Vδ2 T cells and other markers of anti-tumor immunity.

### Multiplex Cytokine Assay

Serum cytokine levels (IFNγ, IL-113, IL-2, IL-4, IL-6, IL-8, IL-13, TNFα) were measured using the MSD (Mesoscale Discovery) platform, according to the manufacturer's instructions.

### Flow Cytometry Analysis

For assessment of Target occupancy (TO) following ICT01 dosing, fresh blood samples (100 μI) were mixed with a cocktail of antibodies (50 μI) prepared in PBS 1% FBS and containing anti-CD45, anti-CD3, anti-CD19, an anti-BTN3A ICT01 competing mAb (Clone 20.1), an anti-BTN3A ICT01 non-competing mAb (clone 103.2) and a viability marker. A second mix where anti-BTN3A were replaced by their respective isotype controls was used in parallel. Samples were incubated for 20 minutes at RT in the dark. RBC were lysed by adding 2 mL of pre-warmed 1×RBC lysis buffer (Biolegend #420302), vortexing and adding a further 2 mL of 1×RBC lysis buffer followed by incubating for 15 minutes at RT in the dark. After washes in PBS 1% FBS, the cell pellets were resuspended in 300 μL of PBS 1% FBS and acquired on a BD LSR Fortessa X-20 using a pre-defined application setting on BD FACS Diva software (v8.0). Data were normalized with the equation: ((MFI test-MFI IC)/(MFI test pre-dose-MFI IC pre-dose)×100). Immunophenotyping was performed using the same procedure with a validated 13 colour panel. The analysis was performed using FlowJo Software (v10.6).

### Immunohistochemistry

Biopsies samples from EVICTION clinical trial were collected pre-treatment and at day 28 post-treatment. At each time point, half of the biopsy was embedded in paraffin and the other half was snap-frozen in optimal cutting temperature compound (OCT). Immunohistochemistry was performed by Veracyte (Marseille, France) on Bond RX Automatic Stainer (Leica Biosystems) and images were acquired using a Nanozoomer XR scanner (Hamamatsu). Briefly, 6 μm thick sections of fresh frozen samples were fixed in zinc formalin for 5 min at RT, incubated 30 min with 5% human serum for blocking and stained with anti-panBTN3A mAb (clone 20.1) or anti-Vγ9TCR mAb (clone 7B6 as described in Huang et al, Infection and Immunity 2008, pp 426-436) at 2 μg/mL for 45 minutes at RT. Antigen-antibody binding was revealed with Bond Polymer Refine RED Detection kit (Leica Biosystems). Tissue sections were then counter-stained with hematoxylin. Multiplex immunohistochemistry stainings for BTN3A2, BTN3A3, δ TCR (clone H-41), CD3, CD4, FoxP3, Ki67, CD8, NKp46, and Granzyme B were performed using the Brightplex from Veracyte on a single FFPE slide. Briefly, 4 μm slides from paraffin embedded biopsies were dewaxed, treated for 20 min with Epitope Retrieval Solution I (Leica Biosystems) and successively stained with indicated mAbs. Antigen-antibody binding was revealed using MACH 2 HRP Polymer (Biocare). Tissue sections were then stained with ImmPACT™ AMEC Red (Vector Lab), and counter-stained with hematoxylin. Between each staining cycle, slides were AMEC destained by ethanol and antibodies were stripped with denaturing solution. Images were acquired between each cycle of staining.

### Results

1. Baseline γδ T Cell Counts Correlate with Anti-BTN3A Antibody Mediated Activation of CD8 T Cells, NK Cells and Granulocytes in Peripheral Blood

Blood samples from EVICTION patients were obtained, prior to treatment (baseline), and 30 minutes, 1, 7 and 21 days post-ICT01 infusion. Immunophenotyping was performed to assess the absolute number and frequency of granulocytes, monocytes, B cells, NK cells and T cells (including CD4 and CD8 ab T cell and Vγ9Vδ2 T cell subsets) as well as their activation status (CD69 and/or PDL-1 surface expression).

In all patients, ICT01 induced a drop in both number and frequency of Vγ9Vδ2 T cells as early as 4h post dose. Vγ9Vδ2 T cells levels progressively returned towards baseline between day 7 and day 21 in patients that received 0.07 to 20 mg ICT01 whereas they remain very low in patients dosed with doses 75 mg. Assessment of CD69 surface expression showed that Vγ9Vδ2 T cells harbor an activated profile post ICT01 dosing in most ICT01-treated patients. In patients that received mg ICT01, NK, conventional γδ T cells and B cell numbers constantly decrease 30 minutes post-dosing, remain low compared to baseline one day later and are restored close to baseline at day 7 in all cohorts. This decrease is associated with activated profile (as assessed by CD69 surface staining) for NK and CD8 T cells in a lower extent. In addition, increase PDL-1 expression on granulocytes is consistently observed in patients dosed with mg ICT01.

When correlated (Spearman rank) with the baseline number of the different immune subsets assessed in this study, NK, CD8 T cells and granulocytes activation appeared to be significantly associated with the baseline number of circulating Vγ9Vδ2 T cells but not with other immune compartments suggesting a secondary effect of ICT01-mediated activation of Vγ9Vδ2 T cells.

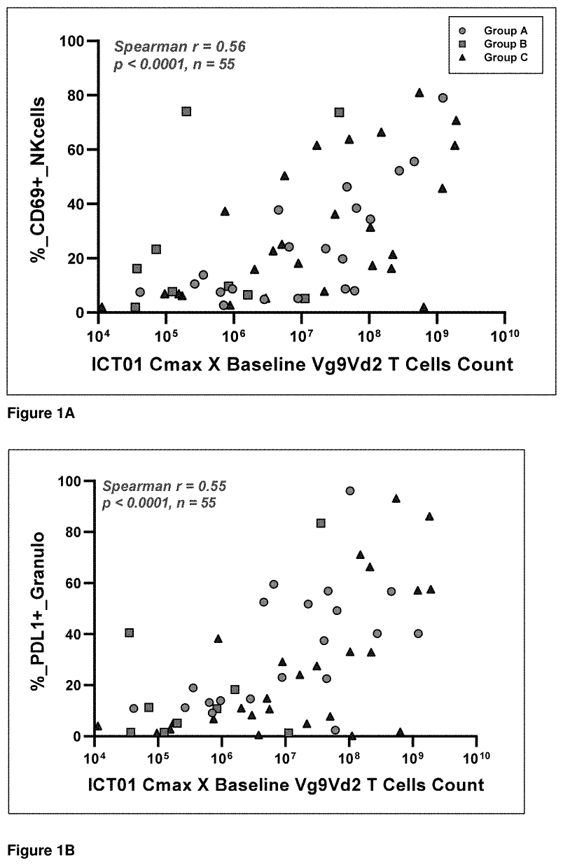

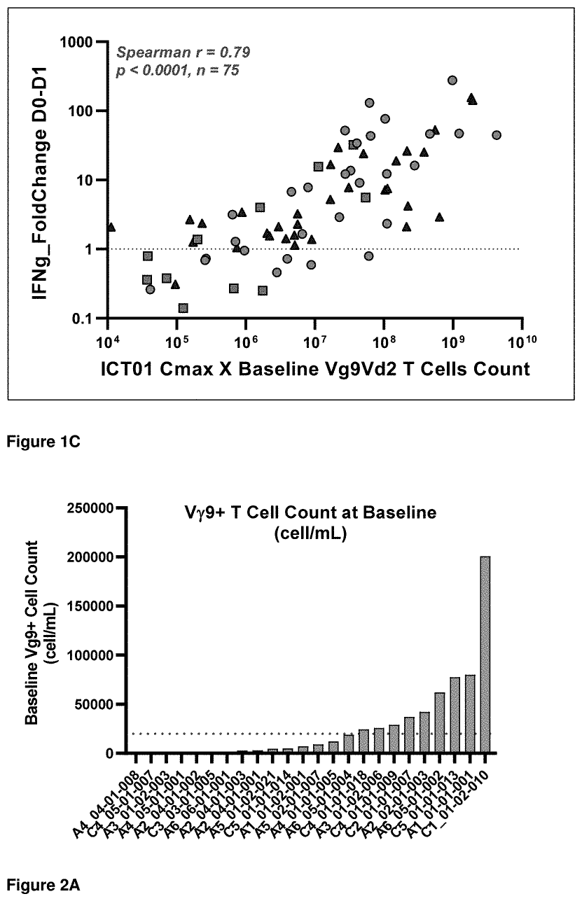

In 55 patients of groups A, B and C from the EVICTION trial, activation of NK cells (CD69 positivity) post ICT01 was strongly related to both ICT01 exposure and baseline g9d2 T Cell Count (spearman r=0.56, p<0.0001) (see FIG. 1A).

Activation of granulocytes (PD-L1 positivity) was also strongly related to both ICT01 exposure and baseline γ9δ2 T Cell Count (spearman r=0.55, p<0.0001) (see FIG. 1B).

2. Baseline γδ T Cell Counts Correlate with In Vivo Anti-BTN3A Antibody Mediated Production of Cytokines

Circulating concentrations of cytokines were quantified using the MSD plateform in the serum of EVICTION patient prior treatment and 30 minutes, 4 hours, 1, 7 and 21 days post-ICT01 treatment. Peak concentrations were observed at 4 h post ICT01 dosing for most cytokines, except TNFa for which the peak was observed at 30 min post ICT01 dosing.

Strong relations between circulating γ9δ2 T cell absolute counts at baseline and serum cytokines and chemokines concentrations post ICT01 dosing were observed for IFNγ, TNFα, IL-6, IL-8 and IL-1b, IL-4, IL-13, and IL-12.

In 75 patients of groups A, B and C from the EVICTION trial, increase in circulating IFNg levels in the first 24h post ICT01 dosing was strongly related to both ICT01 exposure and baseline γ9δ2 T Cell Count (spearman r=0.79, p<0.0001) (see FIG. 1C).

3. Baseline γδ T Cell Counts is Associated with Anti-BTN3A Antibody Mediated Immune Tumor Infiltration and Activation in the Tumor

Tumoral immune infiltration was evaluated in pre-treatment and post-treatment tumor biopsies using both multiplex IHC coupled with digital pathology (Brightplex plateform, HalioDX, Luminy, France) and gene expression profiling (nCounter NanoString platform).

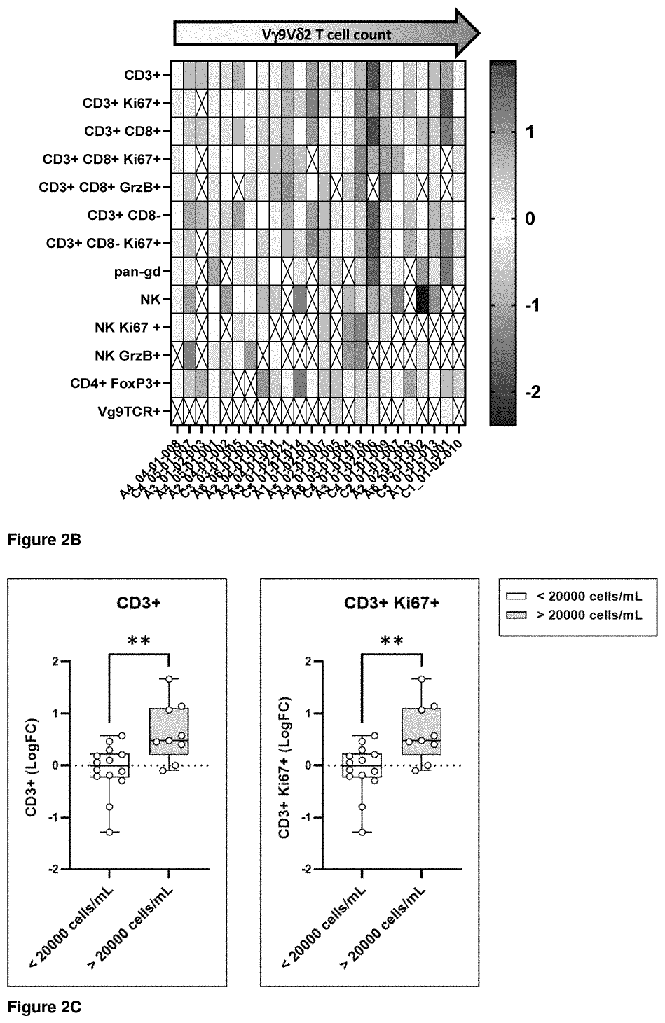

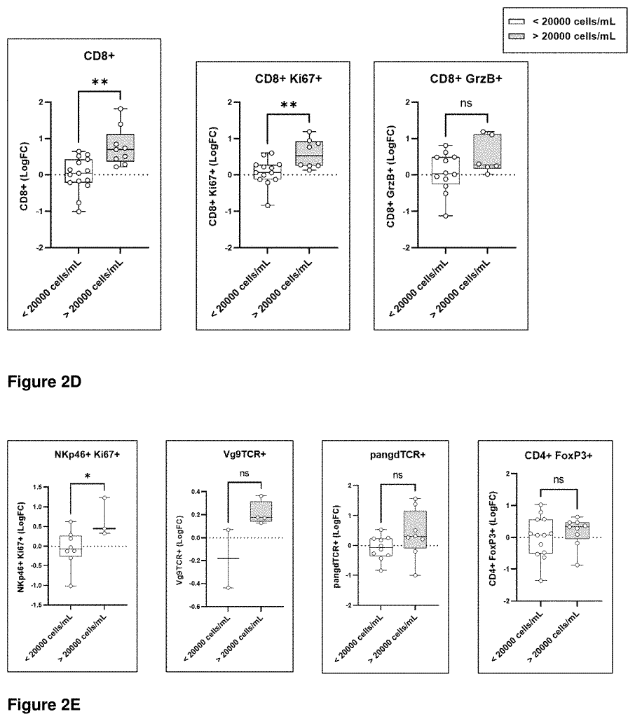

When comparing changes in cell densities of different immune cells populations in post-treatment vs pre-treatment biopsies, patients with highest peripheral blood Vgamma9 T cells absolute counts at baseline trend toward increased immune infiltration and activation in post-treatment biopsies (see FIGS. 2A, 2B, 2C, 2D and 2E).

Those results were confirmed by gene expression analysis using the NanoString plateform. A clear trend for higher immune infiltration and activation in post-treatment biospsies was observed in patient with high Vgamma9 T cells counts at baseline (Not shown).

4. Baseline Gd T Cell Counts Correlate with Vg9+ T Cell Density in the Tumor

Blood samples from EVICTION patients were obtained prior to treatment (baseline). Immunophenotyping was performed to assess the absolute number Vγ9Vδ2 T cells. Vγ9TCR-positive T cell density was evaluated in pre-treatment biopsy using IHC on fresh frozen section coupled with digital pathology (HalioDX, Luminy, France).

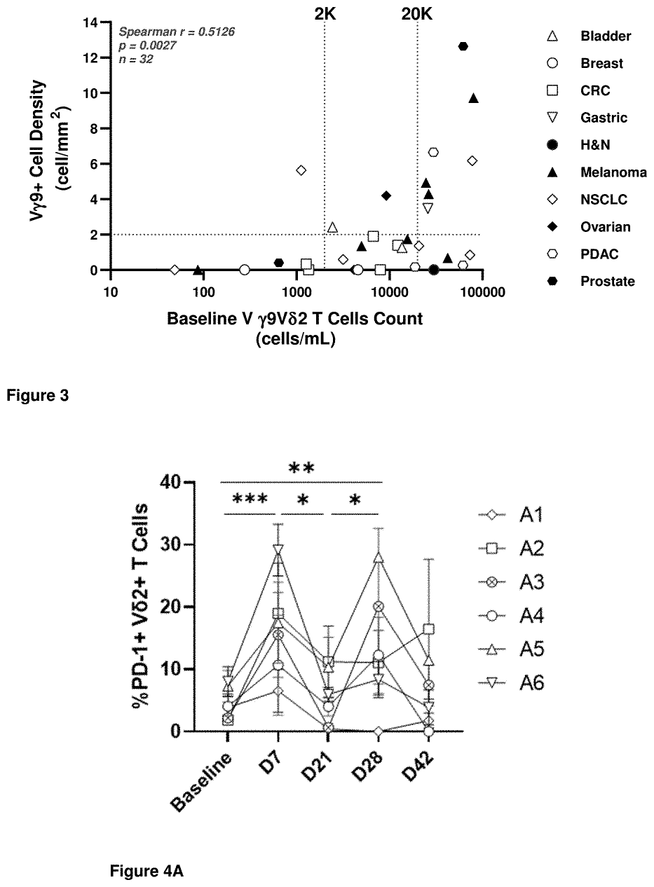

A trend for correlation (Spearman r=0.5126, p=0.0027, n=32) between blood Vγ9T+ cell absolute count at baseline and Vγ9+ Cell Density in the baseline tumor biopsy was observed (see FIG. 3), suggesting that at baseline, Vγ9 tumoral infiltration is related to the absolute count of that subset in the blood.

### Example 3: Clinical Evidence of Treatment with ICT01 in Combination with Anti-PD1 Antibody of Solid Tumors in Patients Refractory or Relapsed Tumors after Anti-PD1/PDL1 Treatment

1. ICT01 Increases PD-1 Expression in EVICTION Patients

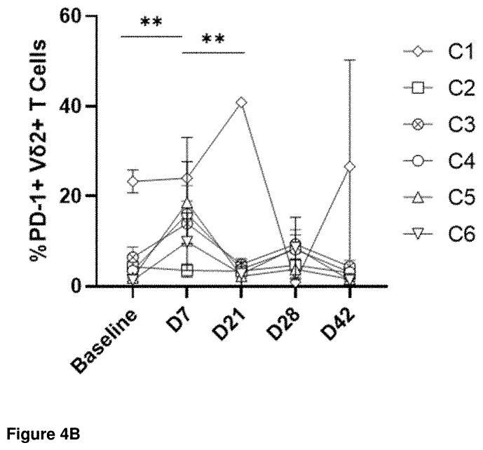

Flow cytometry on frozen biopsies demonstrated that ICT01-induced activation of γ9δ2 T cells increases surface expression of PD-1 (mean per dose cohort are depicted) in cancer patients treated in Group A (FIG. 4A) and Group B (FIG. 4B) of EVICTION.

Two-way ANOVA and Holm-Šidák's multiple comparisons test

2. Activation and Migration of Multiple Immune Cell Populations Post ICT01/Pembrolizumab Treatment

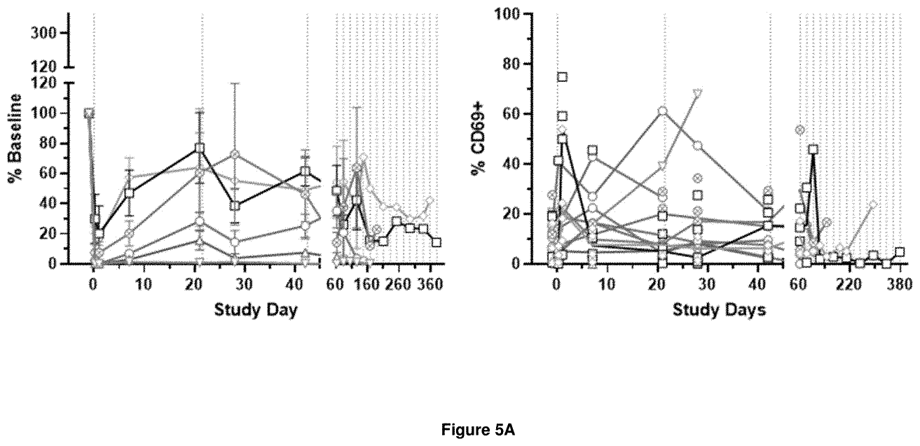

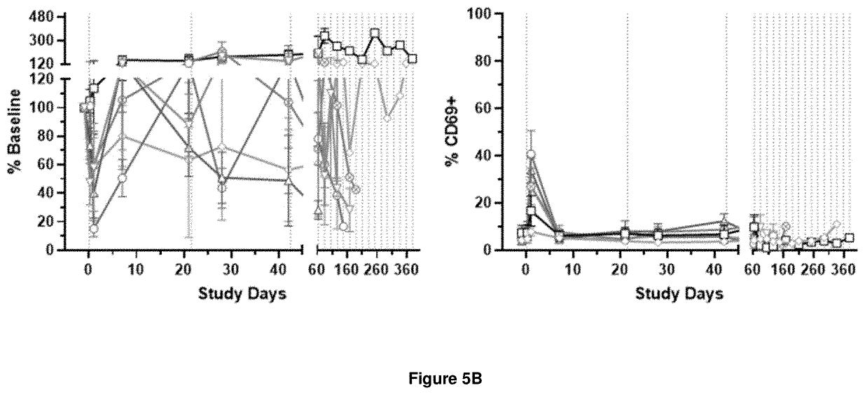

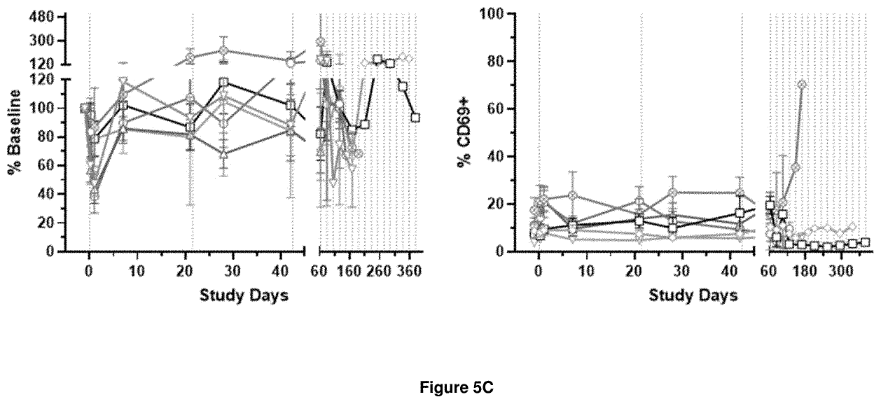

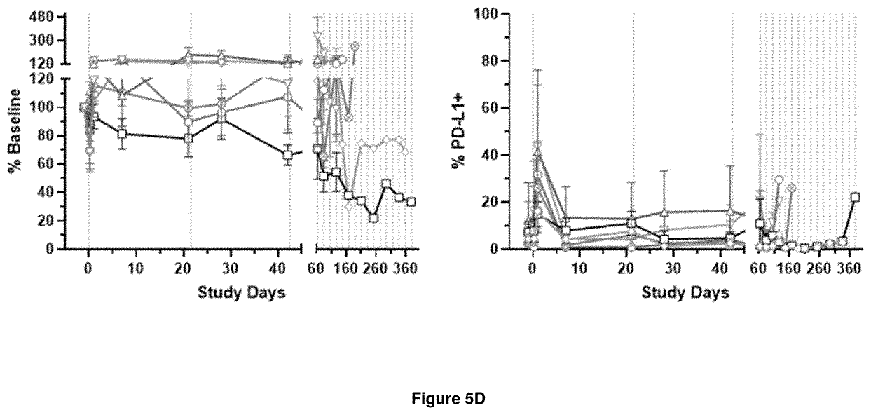

In EVICTION group C, flow cytometry analyses demonstrated that γ9 δ2T cells migrate rapidly post dose with nearly 100% migration observed at all doses tested, with the duration of effect being dose dependent (absolute cell number, % of baseline FIG. 5A, left panel), with increase in activation at 24h (% of CD69 positivity, FIG. 5A, right panel).

Similar pharmacodynamic effects on NK cells and CD8 T cells are observed at 7 mg and higher, with peak effects observed at 75 mg (FIGS. 5B and 5C, respectively).

Activation of granulocytes was observed at all doses, although the migration out of the blood was minimal (FIG. 5D).

The peak effect on γ9 δ2T cells was observed 30 minutes post treatment, while the peak effect on CD8 and NK cells was observed at 24 hours post treatment. These data suggest that there is a step after γ9 δ2T cell activation that is required to create these effects, which we have identified as being cytokine-mediated.

3. Clinical Activity

In the escalation phase of group C of the EVICTION trial, the combination of ICT01 plus pembrolizumab was well tolerated without any DLTs or safety concerns observed.

The most common TEAEs are consistent with an IRR, which is a well-described event for pembrolizumab. No immune-related AESI were reported.

Activation & migration of γ9 δ2T cells in the blood was observed at all ICT01 doses within 30 minutes post dose. In addition, activation & migration of CD8 T cells and NK cells in the blood were observed at doses ≥7 mg ICT01 and appear to be mediated by release of IFNγ and TNFα from activated γ9 δ2T cells. Peripheral immune activation was reflected by infiltration of tumors by γδ, CD3 and CD8 T cells.

Clinical responses in these CPI failure patients were observed across multiple different solid tumors at ICT01 doses as low as 2 mg, suggesting that the complementary mechanisms of action lead to an increased anti-tumor immune response.

### Example 4: Additional Clinical Evidence of Treatment with ICT01 in Combination with Anti-PD1 Antibody in PD1 Refractory Cancer Patients

### Materials and Methods

### Patients and Clinical Trial

EVICTION (NCT04243499) is a first-in-human, two-part, open-label, clinical study to assess the safety, tolerability, pharmacokinetics, pharmacodynamics and anti-tumor activity of IV doses of ICT01 as monotherapy and in combination with pembrolizumab, in patients with advanced-stage, relapsed/refractory cancer. Adult patients must have failed all available standard of care for their cancer to be eligible, and not received other anti-cancer therapy during the trial, except patients in the combination arm of the trial. Patients receive ICT01 (Range: 20 μg to 200 mg) every 3 weeks with blood samples collected at multiple timepoints for safety measures, immunophenotyping on whole blood and serum cytokine analysis.

Tumor biopsies were collected at baseline and on day 28 and stained by immunohistochemistry for Vγ9Vδ2 T cells and other markers of anti-tumor immunity.

### Immunohistochemistry

Biopsies samples from EVICTION clinical trial were collected pre-treatment and at day 28 post-treatment. At each time point, half of the biopsy was embedded in paraffin and the other half was snap-frozen in optimal cutting temperature compound (OCT). Immunohistochemistry was performed by Veracyte (Marseille, France) on Bond RX Automatic Stainer (Leica Biosystems) and images were acquired using a Nanozoomer XR scanner (Hamamatsu). Briefly, 6 μm thick sections of fresh frozen samples were fixed in zinc formalin for 5 min at RT, incubated 30 min with 5% human serum for blocking and stained with anti-Vγ9TCR mAb (clone 7B6) at 2 μg/mL for 45 minutes at RT. Antigen-antibody binding was revealed with Bond Polymer Refine RED Detection kit (Leica Biosystems). Tissue sections were then counter-stained with hematoxylin. Multiplex immunohistochemistry stainings for PD-L1, LAG3, TIM3, PD-1, δ TCR (clone H-41), CD3, CD4, FoxP3, Ki67, CD8, NKp46, and Granzyme B were performed using the Brightplex from Veracyte on a single FFPE slide. Briefly, 4 μm slides from paraffin embedded biopsies were dewaxed, treated for 20 min with Epitope Retrieval Solution I (Leica Biosystems) and successively stained with indicated mAbs. Antigen-antibody binding was revealed using MACH 2 HRP Polymer (Biocare). Tissue sections were then stained with ImmPACT™ AMEC Red (Vector Lab), and counter-stained with hematoxylin. Between each staining cycle, slides were AMEC destained by ethanol and antibodies were stripped with denaturing solution. Images were acquired between each cycle of staining.

### Results

BTN3A Activating Antibody Treatment Combined with Pembrolizumab Induces Clinical Response in Late-Stage, PD-1 Refractory Cancer Patients

A total of 144 patients were dosed with ICT01 in EVICTION (87 ICT01 monotherapy and 57 ICT01 in combination with pembrolizumab). No dose limiting toxicity was observed, with similar safety in both solid and hematologic indications. Safety was also similar in ICT01 monotherapy compared to the pembrolizumab combo group. The main adverse event was infusion-related reaction, normally only related to 1st dose. Few ≥ Grade 3 (severe) treatment-related adverse events were observed (7.53% of all events, in 24 pts, 19%), with IRR (7 events in 6 patients), lymphopenia (n=4 in 4 pts), neutropenia (n=4 in 3 pts), liver enzymes increased (n=2 in 2 pts) and isolated events: acute pulmonary oedema, diabetes, TIA, hypertension.

40 patients were enrolled in the pembrolizumab combination arm of the escalation phase of the EVICTION trial. No new safety signal was identified. The disease control rate (>stable disease at ≥Wk 8 by RECIST1.1) was 42% in melanoma patients (5/12), 22% in NSCLC patients (4/18); 22% in Bladder patients (2/9) and 100% in HNSCC (1/1).

| Patient ID | | | Baseline | | |
|---|---|---|---|---|---|
| ICT01 Dose, | Cancer | | γ9δ2 T | Baseline Sum of | Best |
| Start Date | Dx Date | Prior Therapies | Cells | Target Lesions (n) | Response |
| 01-01-007 | Melanoma | 1. IPI/NIVO April 2020-December 2020 | 37065 | 58 (2) | PR W16-96 |
| 2 mg | (2019) | 2. MEKtovi January 2021-April 2021 | | | (CR brain |
| | | | | | met) |
| 14 Apr. 2021 | | | | | (−71%) |
| 01-01-018 | Melanoma | 1. IPI/NIVO June 2021-October 2021 | 24611 | 104 (1) | PR W16-64 |
| 20 mg | (2019) | | | | (−42%) |
| 4 Jan. 2022 | | | | | |
| 05-01-008 | Melanoma | 1. IPI/NIVO April 2021-July 2021 | 6689 | 66 (3) | SD W56 |
| 200 mg | (2021) | 2. NIVO September 2021-October 2021 | | | (−23%) |
| 2 Feb. 2022 | | | | | |
| 01-01-026 | Melanoma | 1. PEMBRO April 2020-April 2020 | 3192 | 70 (3) | SD W16 |
| 200 mg | (2019) | 2. IPI/NIVO October 2021-December 2021 | | | (−21%) |
| 14 Feb. 2022 | | 3. PEMBRO + TLR3 Agonist June 2021-September 2021 | | | |
| 04-01-029 | Melanoma | 1. IFN August 2013-February 2015 | 6971 | 46 (2) | SD W16 |
| 200 mg | (2013) | 2. PEMBRO May 2020-August 2020 | | | (−2%) |
| 23 Mar. 2022 | | 3. PEMBRO June 2019-September 2019 | | | |
| | | 4. NIVO November 2021-February 2022 | | | |
| 01-01-008 | NSCLC | 1. Cis & Pemetrexed November 2017-April 2018 | 3175 | 15 (1) | PR W16/24 |
| 7 mg | (2017) | 2. NIVO April 2018-October 2020 | | | |
| 5 Jul. 2021 | | | | | |
| 04-01-014 | NSCLC | 1. Carbo/Pemetrexed/PEMBRO February 2019- | 7161 | 44 (2) | SD W24 |
| 2 mg | | April 2019 | | | |
| 27 Apr. 2021 | (2013) | 2. Pemetrexed May 2019-November 2019 | | | |
| | | 3. PEMBRO May 2019-April 2020 | | | |
| | | 3. Docetaxel/Vangatef June 2020-February 2021 | | | |
| 01-01-009 | NSCLC | 1. Carbo/Taxol 2019 | 29123 | 95 (1) | SD W16 |
| 20 mg | (2019) | 2. 4. Taxol February 2021-March 2021 | | | |
| 8 Sep. 2021 | | 3. PEMBRO March 2020-September 2020 | | | |
| | | 4. Vinorelbine April 2021-July 2021 | | | |
| | | 5. Gem September 2020-February 2021 | | | |
| 02-01-012 | NSCLC | 1. Cis/Pemetrexed February 2020-4/2 | 1123 | 65 (2) | SD W8 |
| 20 mg | (2020) | 2. Atezolizumab February 2021-August 2021 | | | |
| 23 Sep. 2021 | | 3. Cis/Vinorelbine May 20-June 2020 | | | |
| 04-01-007 | | 1. Gem/Cis November 2015-January 2016 | 2447 | 11 (1) | PR W16-48 |
| 700 mcg/2 mg/ | Bladder | 2. NIVO October 2018-November 2019 (PD) | | | |
| 7 mg | (2015) | 3. Gem/Cis November 2016-January 2017 | | | |
| 22 Feb. 2021 | | 4. Gem/Carbo February 20-June 2020 | | | |
| 01-02-028 | Bladder | 1. Ameticyne 2012 | 514 | 16 (1) | SD W8/16 |
| 200 mg | (2012) | 2. Gem/Cis January 2019-?/2019 | | | |
| 26 Jan. 2022 | | 3. Gem/Cis February 2018-May 2018 | | | |
| | | 4. PEMBRO October 2020-December 2021 | | | |
| 01-01-015 | HNSCC | 1. Cis/5-FU/Cetuximab March 2018-July 2018 | 29998 | 40 (1) | SD W8 |
| 75 mg | (2011) | 2. Carbo/Taxol/Olaparib January 20-March 2021 | | | |
| 21 Dec. 2021 | | 3. NIVO January 2019-unk/ | | | |
| | | 4. Investigational anti-integrin + ADC | | | |
| | | April 2021-November 2021 | | | |

BTN3A Activating Antibody Treatment Mediated Strong TME Remodelling with Increased Immune Infiltration and Proliferation in the Tumor

An extensive analysis of the changes in CD8 T cells infiltration, activation and exhaustion was performed in pre-treatment and post-treatment tumor biopsies using multiplex IHC coupled with digital pathology (Brightplex plateform, Veracyte, Luminy, France).

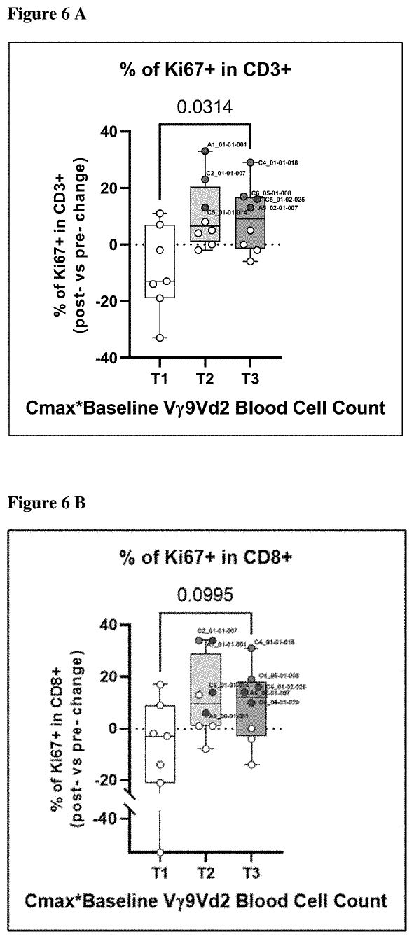

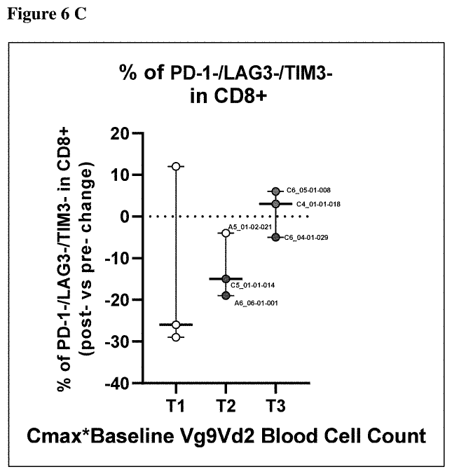

Good quality pre-vs post-treatment biopsy pairs were obtained from 26 patients from group A and C of the escalation phase of the EVICTION study. When comparing changes in the % of Ki67+ in CD3+ cells (FIG. 6A) or CD8+ cells (FIG. 6 B) in post-treatment vs pre-treatment biopsies, a clear trend toward increased immune proliferation was observed in patients in the highest tertile of the ICT01 Cmax X baseline circulating Vγ9Vδ2 T cells count composite and in clinical responders from the pembrolizumab combination group (C2_01-01-007, C4_01-01-018, and C6_05-01-008).

Furthermore, when comparing changes in the % of cells triple positive for PD-1, LAGS and TIM3 in CD8+ cells (FIG. 6 C) in post-treatment vs pre-treatment biopsies, a clear trend toward decreased immune exhaustion was observed in patients in the highest tertile of the ICT01 Cmax X baseline circulating Vγ9Vδ2 T cells count composite and in clinical responders from the pembrolizumab combination group (C4_01-01-018 and C6_05-01-008).

BTN3A Activating Antibody Treatment Induces Strong TME Remodelling with Increased PD-L1 Expression

Analysis of the changes in PD-L1 expression in both the tumor cells and the immune cells was performed in pre-treatment and post-treatment tumor biopsies using multiplex IHC coupled with semi-quantitative evaluation by a trained pathologist (Brightplex plateform, Veracyte, Luminy, France).

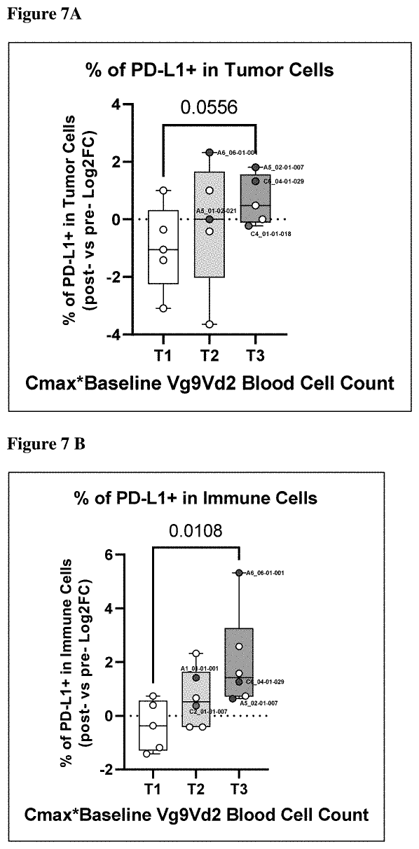

Good quality pre-vs post-treatment biopsy pairs were obtained from 26 patients from group A and C of the escalation phase of the EVICTION study. When comparing changes in the levels of expression of PD-L1 in tumor cells (FIG. 7A) or immune cells (FIG. 7 B) in post-treatment vs pre-treatment biopsies, a clear trend toward increased PD-L1 expression was observed in patients in the highest tertile of the ICT01 Cmax X baseline circulating Vγ9Vδ2 T cells count composite.

### Example 45: Useful Sequences for Practicing the Invention

TABLE 5
Brief description of useful amino acid and nucleotide
sequences for practicing the invention

| SEQ ID NO: | Type | Description of the sequence |
|---|---|---|
| 1 | aa | Humanized heavy chain variable region VH2 of mAb 7.2 |
| 2 | aa | Humanized light chain variable region V□1 of mAb 7.2 |
| 3 | aa | Humanized light chain variable region V□2 of mAb 7.2 |
| 4 | aa | Full length heavy chain of mAbs 1 and 2 (VH2 7.2 silent IgG1) |
| 5 | aa | Full length heavy chain of mAb 3 and 4 (VH2 7.2 silent IgG4) |
| 6 | aa | Full length light chain of mAbs 1 and 3 (Vk1 7.2) |
| 7 | aa | Full length light chain of mAbs 2 and 4 (Vk2 7.2) |
| 8 | nt | Full length heavy chain of mAbs 1 and 2 (VH2 7.2 silent IgG1) |
| 9 | nt | Full length heavy chain of mAbs 3 and 4 (VH2 7.2 silent IgG4) |
| 10 | nt | Full length light chain of mAbs 1 and 3 (Vk1 7.2) |
| 11 | nt | Full length light chain of mAbs 2 and 4 (Vk2 7.2) |
| 12 | aa | HCDR1 of mAb 7.2, 1, 2, 3 and 4 |
| 13 | aa | HCDR2 of mAb 7.2, 1, 2, 3 and 4 |
| 14 | aa | HCDR3 of mAb 7.2, 1, 2, 3 and 4 |
| 15 | aa | LCDR1 of mAb 7.2, 1, 2, 3 and 4 |
| 16 | aa | LCDR2 of mAb 7.2, 1, 2, 3 and 4 |
| 17 | aa | LCDR3 of mAb 7.2, 1, 2, 3 and 4 |
| 18 | aa | Human BTN3A1 |
| 19 | aa | Human BTN3A2 |
| 20 | aa | Human BTN3A3 |
| 21 | aa | Cynomolgus macaque (m. fascicularis) BTN3A1 ectodomain |
| | | used for recombinant protein production |
| 22 | aa | Cynomolgus macaque (m. fascicularis) BTN3A2 ectodomain |
| | | used for recombinant protein production |
| 23 | aa | Cynomolgus macaque (m. fascicularis) BTN3A3 ectodomain |
| | | used for recombinant protein production |
| 24 | aa | Humanized heavy chain variable region of mAb 20.1 |
| 25 | aa | Humanized light chain variable region of mAb 20.1 |
| 26 | aa | HCDR1 |
| 27 | aa | HCDR2 |
| 28 | aa | HCDR3 |
| 29 | aa | LCDR1 |
| 30 | aa | LCDR2 |
| 31 | aa | LCDR3 |
| 32 | aa | Full length heavy chain of humanized mAb 20.1 |
| 33 | aa | Full length light chain of humanized mAb 20.1 |

TABLE 6
Brief description of useful amino acid and nucleotide sequences
for practicing the invention

| SEQ ID | |
|---|---|
| NO: | Specific Amino acid or Nucleotide sequences |
| 1 | QVQLVQSGAEVKKPGASVKLSCKASGYIFTRYYMYWVKQRPGQGLEWIGEI |
| | NPNNGGTKFNEKFKNRATLTVDKSISTAYMELSRLRSDDTAVYYCSREDDY |
| | DGTPFAMDYWGQGTLVTVSS |
| 2 | DIQMTQSPSSLSASVGDRVTITCHASQNINVWLSWYQQKPGKAPKLLIYKAS |
| | NLHTGVPSRFTGSGSGTDFTFTISSLQPEDIATYYCQQGQTYPYTFGQGTKL |
| | EIK |
| 3 | DIQMTQSPSSLSASVGDRVTITCHASQNINVWLSWYQQKPGKAPKLLIYKAS |
| | NLHTGVPSRFSGSGSGTDFTFTISSLQPEDIATYYCQQGQTYPYTFGQGTKL |
| | EIK |
| 4 | QVQLVQSGAEVKKPGASVKLSCKASGYIFTRYYMYWVKQRPGQGLEWIGEI |
| | NPNNGGTKFNEKFKNRATLTVDKSISTAYMELSRLRSDDTAVYYCSREDDY |
| | DGTPFAMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPEFEGGPSVFLFPPKPKDT |
| | LMISRTPEVTCVVVDVSHEDPEVKFNWYVDGVEVHNAKTKPREEQYNSTYR |
| | VVSVLTVLHQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPG |
| 5 | QVQLVQSGAEVKKPGASVKLSCKASGYIFTRYYMYWVKQRPGQGLEWIGEI |
| | NPNNGGTKFNEKFKNRATLTVDKSISTAYMELSRLRSDDTAVYYCSREDDY |
| | DGTPFAMDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYT |
| | CNVDHKPSNTKVDKRVESKYGPPCPPCPAPEFEGGPSVFLFPPKPKDTLMI |
| | SRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLG |
| 6 | DIQMTQSPSSLSASVGDRVTITCHASQNINVWLSWYQQKPGKAPKLLIYKAS |
| | NLHTGVPSRFTGSGSGTDFTFTISSLQPEDIATYYCQQGQTYPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 7 | DIQMTQSPSSLSASVGDRVTITCHASQNINVWLSWYQQKPGKAPKLLIYKAS |
| | NLHTGVPSRFSGSGSGTDFTFTISSLQPEDIATYYCQQGQTYPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 8 | CAGGTCCAACTGGTGCAGTCTGGGGCTGAAGTGAAGAAGCCTGGGGCT |
| | TCAGTGAAGTTGTCCTGCAAGGCTTCTGGCTACATCTTCACCAGATACTA |
| | TATGTATTGGGTGAAGCAGAGGCCTGGACAAGGCCTTGAGTGGATTGGA |
| | GAGATTAATCCTAACAATGGTGGTACTAAGTTCAATGAGAAGTTCAAGAA |
| | CAGGGCCACACTGACTGTAGACAAATCCATCAGCACAGCATACATGGAG |
| | CTCAGCAGGCTGAGATCTGACGACACGGCGGTCTATTATTGTTCAAGAG |
| | AGGATGATTACGACGGGACCCCCTTTGCTATGGACTACTGGGGTCAAGG |
| | AACCCTGGTCACCGTCTCCTCAGCCTCCACCAAGGGCCCATCGGTCTTC |
| | CCCCTGGCACCCTCCTCCAAGAGCACCTCTGGGGGCACAGCGGCCCTG |
| | GGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTA |
| | CAGTCCTCAGGACTCTACTCCCTCAGCAGCGTGGTGACCGTGCCCTCCA |
| | GCAGCTTGGGCACCCAGACCTACATCTGCAACGTGAATCACAAGCCCAG |
| | CAACACCAAGGTGGACAAGAGAGTTGAGCCCAAATCTTGTGACAAAACT |
| | CACACATGCCCACCGTGCCCAGCACCTGAATTCGAGGGGGGACCGTCA |
| | GTCTTCCTCTTCCCCCCAAAACCCAAGGACACCCTCATGATCTCCCGGA |
| | CCCCTGAGGTCACATGCGTGGTGGTGGACGTGAGCCACGAAGACCCTG |
| | AGGTCAAGTTCAACTGGTACGTGGACGGCGTGGAGGTGCATAATGCCAA |
| | GACAAAGCCGCGGGAGGAGCAGTACAACAGCACGTACCGTGTGGTCAG |
| | CGTCCTCACCGTCCTGCACCAGGACTGGCTGAATGGCAAGGAGTACAA |
| | GTGCAAGGTCTCCAACAAAGCCCTCCCAGCCTCCATCGAGAAAACCATC |
| | TCCAAAGCCAAAGGGCAGCCCCGAGAACCACAGGTGTACACCCTGCCC |
| | CCATCCCGGGAAGAGATGACCAAGAACCAGGTCAGCCTGACCTGCCTG |
| | GTCAAAGGCTTCTATCCCAGCGACATCGCCGTGGAGTGGGAGAGCAAT |
| | GGGCAGCCGGAGAACAACTACAAGACCACGCCTCCCGTGCTGGACTCC |
| | GACGGCTCCTTCTTCCTCTATAGCAAGCTCACCGTGGACAAGAGCAGGT |
| | GGCAGCAGGGGAACGTCTTCTCATGCTCCGTGATGCATGAGGCTCTGCA |
| | CAACCACTACACGCAGAAGAGCCTCTCCCTGTCTCCGGGTTGA |
| 9 | CAGGTCCAACTGGTGCAGTCTGGGGCTGAAGTGAAGAAGCCTGGGGCT |
| | TCAGTGAAGTTGTCCTGCAAGGCTTCTGGCTACATCTTCACCAGATACTA |
| | TATGTATTGGGTGAAGCAGAGGCCTGGACAAGGCCTTGAGTGGATTGGA |
| | GAGATTAATCCTAACAATGGTGGTACTAAGTTCAATGAGAAGTTCAAGAA |
| | CAGGGCCACACTGACTGTAGACAAATCCATCAGCACAGCATACATGGAG |
| | CTCAGCAGGCTGAGATCTGACGACACGGCGGTCTATTATTGTTCAAGAG |
| | AGGATGATTACGACGGGACCCCCTTTGCTATGGACTACTGGGGTCAAGG |
| | AACCCTGGTCACCGTCTCCTCAGCTTCCACCAAGGGCCCATCCGTCTTC |
| | CCCCTGGCGCCCTGCTCCAGGAGCACCTCCGAGAGCACAGCCGCCCTG |
| | GGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTA |
| | CAGTCCTCAGGACTCTACTCCCTCAGCAGCGTGGTGACCGTGCCCTCCA |
| | GCAGCTTGGGCACGAAGACCTACACCTGCAATGTAGATCACAAGCCCAG |
| | CAACACCAAGGTGGACAAGAGAGTTGAGTCCAAATATGGTCCCCCATGC |
| | CCACCATGCCCAGCACCTGAGTTCGAGGGGGGACCATCAGTCTTCCTGT |
| | TCCCCCCAAAACCCAAGGACACTCTCATGATCTCCCGGACCCCTGAGGT |
| | CACGTGCGTGGTGGTGGACGTGAGCCAGGAAGACCCCGAGGTCCAGTT |
| | CAACTGGTACGTGGATGGCGTGGAGGTGCATAATGCCAAGACAAAGCC |
| | GCGGGAGGAGCAGTTCAACAGCACGTACCGTGTGGTCAGCGTCCTCAC |
| | CGTCCTGCACCAGGACTGGCTGAACGGCAAGGAGTACAAGTGCAAGGT |
| | CTCCAACAAAGGCCTCCCGTCCTCCATCGAGAAAACCATCTCCAAAGCC |
| | AAAGGGCAGCCCCGAGAGCCACAGGTGTACACCCTGCCCCCATCCCAG |
| | GAGGAGATGACCAAGAACCAGGTCAGCCTGACCTGCCTGGTCAAAGGC |
| | TTCTACCCCAGCGACATCGCCGTGGAGTGGGAGAGCAATGGGCAGCCG |
| | GAGAACAACTACAAGACCACGCCTCCCGTGCTGGACTCCGACGGCTCCT |
| | TCTTCCTCTACAGCAGGCTAACCGTGGACAAGAGCAGGTGGCAGGAGG |
| | GGAATGTCTTCTCATGCTCCGTGATGCATGAGGCTCTGCACAACCACTA |
| | CACACAGAAGAGCCTCTCCCTGTCTCTGGGTTGA |
| 10 | GACATCCAGATGACCCAGTCTCCATCCAGTCTGTCTGCATCCGTAGGAG |
| | ACAGAGTCACCATCACTTGCCATGCCAGTCAGAACATTAATGTTTGGTTA |
| | TCTTGGTACCAGCAGAAACCAGGAAAAGCCCCTAAACTCTTGATCTATAA |
| | GGCTTCCAACTTGCACACAGGCGTCCCATCAAGATTTACTGGCAGTGGA |
| | TCTGGAACAGATTTCACATTCACCATCAGCAGCCTGCAGCCTGAAGACAT |
| | TGCCACTTACTACTGTCAACAGGGTCAAACTTATCCATACACGTTCGGAC |
| | AGGGGACCAAGCTGGAGATCAAACGAACTGTGGCTGCACCATCTGTCTT |
| | CATCTTCCCGCCATCTGATGAGCAGTTGAAATCTGGAACTGCCTCTGTTG |
| | TGTGCCTGCTGAATAACTTCTATCCCAGAGAGGCCAAAGTACAGTGGAA |
| | GGTGGATAACGCCCTCCAATCGGGTAACTCCCAGGAGAGTGTCACAGA |
| | GCAGGACAGCAAGGACAGCACCTACAGCCTCAGCAGCACCCTGACGCT |
| | GAGCAAAGCAGACTACGAGAAACACAAAGTCTACGCCTGCGAAGTCACC |
| | CATCAGGGCCTGAGCTCGCCCGTCACAAAGAGCTTCAACAGGGGAGAG |
| | TGTTAG |
| 11 | GACATCCAGATGACCCAGTCTCCATCCAGTCTGTCTGCATCCGTAGGAG |
| | ACAGAGTCACCATCACTTGCCATGCCAGTCAGAACATTAATGTTTGGTTA |
| | TCTTGGTACCAGCAGAAACCAGGAAAAGCCCCTAAACTCTTGATCTATAA |
| | GGCTTCCAACTTGCACACAGGCGTCCCATCAAGATTTAGTGGCAGTGGA |
| | TCTGGAACAGATTTCACATTCACCATCAGCAGCCTGCAGCCTGAAGACAT |
| | TGCCACTTACTACTGTCAACAGGGTCAAACTTATCCATACACGTTCGGAC |
| | AGGGGACCAAGCTGGAGATCAAACGAACTGTGGCTGCACCATCTGTCTT |
| | CATCTTCCCGCCATCTGATGAGCAGTTGAAATCTGGAACTGCCTCTGTTG |
| | TGTGCCTGCTGAATAACTTCTATCCCAGAGAGGCCAAAGTACAGTGGAA |
| | GGTGGATAACGCCCTCCAATCGGGTAACTCCCAGGAGAGTGTCACAGA |
| | GCAGGACAGCAAGGACAGCACCTACAGCCTCAGCAGCACCCTGACGCT |
| | GAGCAAAGCAGACTACGAGAAACACAAAGTCTACGCCTGCGAAGTCACC |
| | CATCAGGGCCTGAGCTCGCCCGTCACAAAGAGCTTCAACAGGGGAGAG |
| | TGTTAG |
| 12 | RYYMY |
| 13 | EINPNNGGTKFNEKFKN |
| 14 | EDDYDGTPFAMDY |
| 15 | HASQNINVWLS |
| 16 | KASNLHT |
| 17 | QQGQTYPYT |
| 18 | MKMASFLAFLLLNFRVCLLLLQLLMPHSAQFSVLGPSGPILAMVGEDADLPC |
| | HLFPTMSAETMELKWVSSSLRQVVNVYADGKEVEDRQSAPYRGRTSILRD |
| | GITAGKAALRIHNVTASDSGKYLCYFQDGDFYEKALVELKVAALGSDLHVDV |
| | KGYKDGGIHLECRSTGWYPQPQIQWSNNKGENIPTVEAPVVADGVGLYAV |
| | AASVIMRGSSGEGVSCTIRSSLLGLEKTASISIADPFFRSAQRWIAALAGTLP |
| | VLLLLLGGAGYFLWQQQEEKKTQFRKKKREQELREMAWSTMKQEQSTRVK |
| | LLEELRWRSIQYASRGERHSAYNEWKKALFKPADVILDPKTANPILLVSEDQ |
| | RSVQRAKEPQDLPDNPERFNWHYCVLGCESFISGRHYWEVEVGDRKEWHI |
| | GVCSKNVQRKGWVKMTPENGFWTMGLTDGNKYRTLTEPRTNLKLPKPPKK |
| | VGVFLDYETGDISFYNAVDGSHIHTFLDVSFSEALYPVFRILTLEPTALTICPA |
| 19 | MKMASSLAFLLLNFHVSLLLVQLLTPCSAQFSVLGPSGPILAMVGEDADLPC |
| | HLFPTMSAETMELKWVSSSLRQVVNVYADGKEVEDRQSAPYRGRTSILRD |
| | GITAGKAALRIHNVTASDSGKYLCYFQDGDFYEKALVELKVAALGSNLHVEV |
| | KGYEDGGIHLECRSTGWYPQPQIQWSNAKGENIPAVEAPVVADGVGLYEV |
| | AASVIMRGGSGEGVSCIIRNSLLGLEKTASISIADPFFRSAQPWIAALAGTLPI |
| | LLLLLAGASYFLWRQQKEITALSSEIESEQEMKEMGYAATEREISLRESLQEE |
| | LKRKKIQYLTRGEESSSDTNKSA |
| 20 | MKMASSLAFLLLNFHVSLFLVQLLTPCSAQFSVLGPSGPILAMVGEDADLPC |
| | HLFPTMSAETMELRWVSSSLRQVVNVYADGKEVEDRQSAPYRGRTSILRD |
| | GITAGKAALRIHNVTASDSGKYLCYFQDGDFYEKALVELKVAALGSDLHIEVK |
| | GYEDGGIHLECRSTGWYPQPQIKWSDTKGENIPAVEAPVVADGVGLYAVAA |
| | SVIMRGSSGGGVSCIIRNSLLGLEKTASISIADPFFRSAQPWIAALAGTLPISLL |
| | LLAGASYFLWRQQKEKIALSRETEREREMKEMGYAATEQEISLREKLQEELK |
| | WRKIQYMARGEKSLAYHEWKMALFKPADVILDPDTANAILLVSEDQRSVQR |
| | AEEPRDLPDNPERFEWRYCVLGCENFTSGRHYWEVEVGDRKEWHIGVCS |
| | KNVERKKGWVKMTPENGYWTMGLTDGNKYRALTEPRTNLKLPEPPRKVGI |
| | FLDYETGEISFYNATDGSHIYTFPHASFSEPLYPVFRILTLEPTALTICPIPKEV |
| | ESSPDPDLVPDHSLETPLTPGLANESGEPQAEVTSLLLPAHPGAEVSPSATT |
| | NQNHKLQARTEALY |
| 21 | MGSSLAFLLLSFHVCVLLLQLLMPHSAQFAVVGPPGPILAMVGEDADLPCHL |
| | FPTMSAETMELRWVSSNLRQVVNVYADGKEVEDRQSAAYRGRTSILRDGIT |
| | AGKAALRIHNVTASDSGKYLCYFQDGDFYEKALVELKVAALGSDLHIDVKGY |
| | EDGGIHLECRSTGWYPQPQIRWSNDKGENIPAVEAPVFVDGVGLYAVAASV |
| | ILRGSSGEGVSCTIRSSLLGLEKTTSISIAG HHHHHH |
| 22 | MGSSLAFLLLNFHVSFFLVQLLTPCSAQFSVLGPSGPILAMVGEDADLPCHL |
| | FPTMSAETMELRWVSSSLRQVVNVYADGKEVEDRQSAPYRGRTSILRDDIA |
| | AGKAALRIHNVTASDSGKYLCYFQDADFYEKALVELKVAALGSNLHVEVKGY |
| | EDGGIHLECRSTGWYPQPKIQWSNAKGQNIPAVEAPVVADGVGLYAVAASV |
| | IMRGGSGESVSCIIRNSVLGLEKTASISIAD HHHHHH |
| 23 | MANFLAFLLLNFRVCLLLVQLLTPCSAQFAVLGPHGPILAMVGEDVDLPCHL |
| | FPTMSAETMELRWVSSSLRQVVNVYSDGKEVEDRQSAPYRGRTSILRDGIT |
| | AGKAALRIHNVTASDSGKYLCYFQDGDFYEKALVELKVAALGSDLHIEVKGY |
| | EDGGIHLECRSTGWYPQPQIQWSNTKGQHIPAVKAPVVADGVGLYAVAASV |
| | IMRGSSGEGVSCIIRNSLLGLEKTASISITD HHHHHH |
| 24 | QVQLVQSGAEVKKPGASVKVSCKASGYTFTRYYLYWVKQRPGQGLEWIGEINPNN |
| | GGTKFNEKFKSRATMTVDKSTRTTYMELSSLRSEDTAVYYCSREDDYDGTPDAMD |
| | YWGQGTLVTVSS |
| 25 | DIQMTQSPSSLSASVGDRVTITCHASQNINLWLSWYQQKPGKAPKLLIYRASNLHTG |
| | VPSRFSGSGSATDFTFTISSLQPEDIATYYCQQGHSYPYTFGGGTKVDIK |
| 26 | RYYLY |
| 27 | EINPNNGGTKFNEKFKS |
| 28 | EDDYDGTPDAMDY |
| 29 | HASQNINLWLS |
| 30 | RASNLHT |
| 31 | QQGHSYPYT |
| 32 | QVQLVQSGAEVKKPGASVKVSCKASGYTFTRYYLYWVKQRPGQGLEWIGEINPNN |
| | GGTKFNEKFKSRATMTVDKSTRTTYMELSSLRSEDTAVYYCSREDDYDGTPDAMD |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKRVEP |
| | KSCDKTHTCPPCPAPEFEGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALP |
| | ASIEKTISKAKGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 33 | DIQMTQSPSSLSASVGDRVTITCHASQNINLWLSWYQQKPGKAPKLLIYRASNLHTG |
| | VPSRFSGSGSATDFTFTISSLQPEDIATYYCQQGHSYPYTFGGGTKVDIKRTVAAPS |
| | VFIFPPSDEQLKSGTASVVCLLNNFYPREAKVQWKVDNALQSGNSQESVTEQDSKD |
| | STYSLSSTLTLSKADYEKHKVYACEVTHQGLSSPVTKSFNRGEC |

1. A method for treating a tumor in a human subject in need thereof, said method comprising administering a therapeutically efficient amount of an anti-BTN3A antibody which induces the activation of Vγ9Vδ2 T cells, in combination with a therapeutically efficient amount of an anti-PD1/PDL1 treatment, wherein said subject is having relapsed or refractory tumors to anti-PD1/PDL1 treatment.

2. The method of claim 1, wherein said tumor is a solid tumor, in particular selected from the group consisting of bladder cancer, melanoma, non small cell lung cancer, and head and neck squamous cell carcinoma.

3. The method of claim 1, wherein said anti-BTN3A antibody binds to human BTN3A polypeptide with a K_{D} of 10 nM or less, as measured by surface plasmon resonance (SPR).

4. The method of claim 1, wherein said anti-BTN3A antibody cross-reacts to cynomolgus BTN3A with a K_{D} of 100 nM or less, as measured by SPR.

5. The method of claim 1, wherein said anti-BTN3A antibody induces in vitro the activation of Vγ9Vδ2 T cells in human PBMC, with an EC₅₀ below 0.1 mg/mL, as measured by surface expression of the activation markers CD69.

6. The method of claim 1, wherein said anti-BTN3A antibody induces the activation of Vγ9Vδ2 T cells in co-culture with BTN3 expressing cells, with an EC₅₀ below 5 mg/mL, as measured in a degranulation assay.

7. The method of claim 1, wherein said activating anti-BTN3A antibody comprises HCDR1 of SEQ ID NO:12, HCDR2 of SEQ ID NO:13, HCDR3 of SEQ ID NO:14, LCDR1 of SEQ ID NO:15, LCDR2 of SEQ ID NO:16 and LCDR3 of SEQ ID NO:17.

8. The method of claim 1, wherein said anti-BTN3A antibody is a humanized antibody.

9. The method of claim 1, wherein said anti-BTN3A antibody includes at least the following amino acid mutations in the VH framework regions: VSQ; V11L; K12V; R66K; S74F; 175S; E81Q; S82AR; R82BS; R83T; D85E; T87S; L108S; and at least the following amino acid mutations in the Vk framework regions: TSN; V15L; R18T; V19I; K42N; A43I; D70G; F73L; Q100G.

10. The method of claim 1, wherein said anti-BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

11. The method of claim 1, wherein said anti-BTN3A antibody comprises a variable heavy chain (VH) of SEQ ID NO:1 and a variable light chain (VL) of SEQ ID NO:2.

12. The method of claim 1, wherein said anti-BTN3A antibody comprises a variable heavy chain (VH) of SEQ ID NO:1 and a variable light chain (VL) of SEQ ID NO:3.

13. The method of claim 1, wherein said mutant IgG1 constant region is IgG1 triple mutant L247F L248E and P350S.

14. The method of claim 1, wherein said anti-BTN3A antibody comprises either
(i) a heavy chain of SEQ ID NO:4 and a light chain of SEQ ID NO:6;
(ii) a heavy chain of SEQ ID NO:4 and a light chain of SEQ ID NO:7;
(iii) a heavy chain of SEQ ID NO:5 and a light chain of SEQ ID NO:6; or,
(iv) a heavy chain of SEQ ID NO:5 and a light chain of SEQ ID NO:7.

15. The method of claim 1, wherein said anti-BTN3A antibody is administered in combination with a cytokine.

16. The method of claim 1, wherein said cytokine is an IL2 or ID 5 agonist.

17. The method of claim 1, wherein said anti-BTN3A antibody is administered in combination with an immunotherapeutic agent.

18. The method of claim 1, wherein said anti-BTN3A antibody is administered in combination with an anti-PD1/anti-PD-L1 treatment selected from the group consisting of an anti-PD1 or anti-PD-L1 antibody.

19. The method of claim 18, wherein said anti-PD1 or anti-PD-L1 antibody is selected from the group consisting of nivolumab, pembrolizumab, avelumab, durvalumab, cemiplimab, and atezolizumab.

20. The method of claim 18, wherein said anti-PD1 or anti-PD-L1 antibody is pembrolizumab.

21. The method of claim 1, wherein the therapeutic dose of the activating anti-BTN3A antibody is within 7 and 200 mg per administration, preferably within 20 and 75 mg.

22. The method of claim 1, wherein said anti-BTN3A antibody comprises a heavy chain of SEQ ID NO:4 and a light chain of SEQ ID NO:6, and the therapeutic dose of the activating anti-BTN3A antibody is within 7 and 200 mg per administration and wherein said subject is administered with a therapeutically efficient amount of pembrolizumab.

23. The method of claim 1, wherein said anti-BTN3A antibody comprises a heavy chain of SEQ ID NO:4 and a light chain of SEQ ID NO:6, and the therapeutic dose of the activating anti-BTN3A antibody is within 20 and 75 mg.

24. The method of claim 1, wherein said anti-BTN3A antibody is administered intravenously at least twice at a dose comprised between 7 and 200 mg each dose, the second dose being administered at least 15 days after the first dose, typically after about 21 days.

25. The method of claim 1, wherein said anti-BTN3A antibody is administered at a dose selected from 7, 10, 20, 50, 75, 100, 125, 150, 170 or 200 mg.

26. A method for enhancing immune cell infiltration in a tumor of a subject in need thereof, said method comprising administering an efficient amount of an anti-BTN3A antibody comprises a heavy chain of SEQ ID NO:4 and a light chain of SEQ ID NO:6, in combination with an efficient amount of an anti-PD1 or anti-PDL1 agent.

27. The method of claim 26, wherein said an anti-PD1 or anti-PDL1 agent is pembrolizumab.

28. The method of claim 26, wherein said immune cells comprises Vγ9Vδ2 T cells and CD8+ T cells.
